(19) United States
(12) Patent Application Publication
(10) Pub. No.: US 2008/0085902 A1
(43) Pub. Date: Apr. 10, 2008

(54) Combination Of A Vegf Receptor Inhibitor Or With A Chemotherapeutic Agent

(76) Inventors: Guido Bold, Gipf-Oberfrick (CH); Josef Bernhard Brueggen, Riehen (DE); Jerry Min-Jian Huang, Florham Park, NJ (US); Frederick Ray Kinder, Morristown, NJ (US); Heidi Lane, Biel-Benken (CH); Elisabeth Jeanne Latour, Bartenheim-La Chaussee (FR); Paul W. Manley, Arlesheim (GB); Jeanette Marjorie Wood, Biel-Benken (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/573,163

(22) PCT Filed: Sep. 23, 2004
(86) PCT No.: PCT/EP04/10686

§ 371 Date: Feb. 28, 2007

Related U.S. Application Data
(60) Provisional application No. 60/505,250, filed on Sep. 23, 2003.

(51) Int. Cl. A61K 31/501; A61K 31/404; A61P 35/00; A61K 31/44

(52) U.S. Cl. 514/252.01; 514/352; 514/415

(57) ABSTRACT

The present invention relates to a combination therapy for treating patients suffering from proliferative diseases or diseases associated with persistent angiogenesis. The patient is treated with a VEGF inhibitor compound; and one or more chemotherapeutic agents.

The invention relates to a method of preventing or treating proliferative diseases or diseases that are associated with or triggered by persistent angiogenesis in a mammal, particularly a human, with a combination of pharmaceutical agents which comprises:
(a) a vascular endothelial growth factor (VEGF) receptor protein tyrosine kinase inhibitor (VEGF inhibitor); and
(b) one or more chemotherapeutic agents.

The invention further relates to pharmaceutical compositions comprising:
(a) a VEGF inhibitor;
(b) one or more chemotherapeutic agents; and
(c) a pharmaceutically acceptable carrier.

The present invention further relates to a commercial package or product comprising:
(a) a pharmaceutical formulation of a VEGF inhibitor; and
(b) a pharmaceutical formulation of a chemotherapeutic agent for simultaneous, concurrent, separate or sequential use.

The combination partners (a) and (b) can be administered together, one after the other or separately in one combined unit dosage form or in two separate unit dosage forms. The unit dosage form may also be a fixed combination.

### BACKGROUND OF THE INVENTION

In the center of the network regulating the growth and differentiation of the vascular system and its components, both during embryonic development and normal growth and in a wide number of pathological anomalies and diseases, lies the angiogenic factor, known as VEGF or VGEF; originally termed vascular permeability factor (VPF), along with its cellular receptors. See Breier et al., Trends Cell Biol, Vol. 6, pp. 454-456 (1996) and references cited therein.

VEGF is a dimeric, disulfide-linked 46-kDa glycoprotein produced by normal cell lines and tumor cell lines. It is an endothelial cell-specific mitogen, shows angiogenic activity in in vivo test systems, e.g., rabbit cornea, is chemotactic for endothelial cells and monocytes, and induces plasminogen activators in endothelial cells, which are then involved in the proteolytic degradation of extracellular matrix during the formation of capillaries. A number of isoforms of VEGF are known, which show comparable biological activity, but differ in the type of cells that secrete them and in their heparin-binding capacity. In addition, there are other members of the VEGF family, such as placenta growth factor and VEGF-C.

VEGF receptors are transmembranous receptor tyrosine kinases. They are characterized by an extracellular domain with seven immunoglobulin-like domains and an intracellular tyrosine kinase domain. Various types of VEGF receptor are known, e.g., VEGFR-1, VEGFR-2 and VEGFR-3.

A large number of human tumors, especially gliomas and carcinomas, express high levels of VEGF and its receptors. This has led to the hypothesis that the VEGF released by tumor cells could stimulate the growth of blood capillaries and the proliferation of tumor endothelium in a paracrine manner and thus, through the improved blood supply, accelerate tumor growth. Increased VEGF expression could explain the occurrence of cerebral oedema in patients with glioma. Direct evidence of the role of VEGF as a tumor angiogenesis factor in vivo has been obtained from studies in which VEGF expression or VEGF activity was inhibited. This was achieved with antibodies which inhibit VEGF activity, with dominant-negative VEGFR-2 mutants which inhibited signal transduction, or with the use of antisense-VEGF RNA techniques. All approaches led to a reduction in the growth of glioma cell lines or other tumor cell lines in vivo as a result of inhibited tumor angiogenesis.

Angiogenesis is regarded as an absolute prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size.

Three principal mechanisms play an important part in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells which normally line the vessels.

Accruing evidence suggests that VEGF inhibitors are even more efficacious when used in combination with other chemotherapeutic agents. There are both synergistic and additive advantages, both for efficacy and safety. Therapeutic effects of combinations of chemotherapeutic agents with VEGF inhibitors can result in lower safe dosages ranges of each component in the combination.

### SUMMARY OF THE INVENTION

The invention relates to a method of preventing or treating proliferative diseases or diseases that are associated with or triggered by persistent angiogenesis in a mammal, particularly a human, with a combination of pharmaceutical agents which comprises:
(a) a VEGF inhibitor; and
(b) one or more chemotherapeutic agents.

The invention further relates to pharmaceutical compositions comprising:
(a) a VEGF inhibitor;
(b) one or more chemotherapeutic agents; and
(c) a pharmaceutically acceptable carrier.

The present invention further relates to a commercial package or product comprising:
(a) a pharmaceutical formulation of a VEGF inhibitor; and
(b) a pharmaceutical formulation of a chemotherapeutic agent for simultaneous, concurrent, separate or sequential use.

### The Chemotherapeutic Agents

The term “chemotherapeutic agents” is a broad one covering many chemotherapeutic agents having different mechanisms of action. Combinations of some of these with VEGF inhibitors can result in improvements in cancer therapy. Generally, chemotherapeutic agents are classified according to the mechanism of action. Many of the available agents are anti-metabolites of development pathways of various tumors, or react with the DNA of the tumor cells. There are also agents which inhibit enzymes, such as topoisomerase I and topoisomerase II, or which are antimiotic agents.

The term “chemotherapeutic agent” includes, but is not limited to:
i. an aromatase inhibitor;
ii. an antiestrogen, an anti-androgen (especially in the case of prostate cancer) or a gonadorelin agonist;
iii. a topoisomerase I inhibitor or a topoisomerase II inhibitor;
iv. a microtubule active agent, an alkylating agent, an anti-neoplastic anti-metabolite or a platin compound;
v. a compound targeting/decreasing a protein or lipid kinase activity or a protein or lipid phosphatase activity, a further anti-angiogenic compound or a compound which induces cell differentiation processes;
vi. a bradykinin 1 receptor or an angiotensin II antagonist;
vii. a cyclooxygenase inhibitor, a bisphosphonate, a heparanase inhibitor (prevents heparan sulphate degradation), e.g., PI-88, a biological response modifier, preferably a lymphokine or interferons, e.g. interferon γ, an ubiquitination inhibitor or an inhibitor which blocks anti-apoptotic pathways;
viii. an inhibitor of Ras oncogenic isoforms or a farnesyl transferase inhibitor;
ix. a telomerase inhibitor, e.g., telomestatin;
x. a protease inhibitor, a matrix metalloproteinase inhibitor, a methionine aminopeptidase inhibitor, e.g., bengamide or a derivative thereof; or a proteasome inhibitor, e.g., PS-341 (bortezomib/Velcade);
xi. agents used in the treatment of hematologic malignancies or FMS-like tyrosine kinase inhibitors;
xii. an HSP90 inhibitors;
xiii. histone deacetylase (HDAC) inhibitors;
xiv. mTOR inhibitors;
xv. somatostatin receptor antagonists;
xvi. integrin antagonists;
xvii. anti-leukemic compounds;
xviii. tumor cell damaging approaches, such as ionizing radiation:
xix. EDG binders;
xx. anthranilic acid amide class of kinase inhibitors;
xxi. ribonucleotide reductase inhibitors;
xxii. S-adenosylmethionine decarboxylase inhibitors;
xxiii. antibodies against VEGF or VEGFR;
xxiv. photodynamic therapy;
xxv. angiostatic steroids;
xxvi. implants containing corticosteroids;
xxvii. AT1 receptor antagonists; and
xxviii. ACE inhibitors.

The term “aromatase inhibitor”, as used herein, relates to a compound which inhibits the estrogen production, i.e., the conversion of the substrates androstenedione and testosterone to estrone and estradiol, respectively. The term includes, but is not limited to, steroids, especially atamestane, exemestane and formestane; and, in particular, non-steroids, especially aminoglutethimide, roglethimide, pyridoglutethimide, trilostane, testolactone, ketokonazole, vorozole, fadrozole, anastrozole and letrozole. Exemestane is marketed as AROMASIN; formestane as LENTARON; fadrozole as AFEMA; anastrozole as ARIMIDEX; letrozole as FEMARA or FEMAR; and aminoglutethimide as ORIMETEN. A combination of the invention comprising a chemotherapeutic agent which is an aromatase inhibitor is particularly useful for the treatment of hormone receptor positive tumors, e.g., breast tumors.

The term “anti-estrogen”, as used herein, relates to a compound which antagonizes the effect of estrogens at the estrogen receptor level. The term includes, but is not limited to, tamoxifen, fulvestrant, raloxifene and raloxifene hydrochloride. Tamoxifen can be administered in the form as it is marketed, e.g., NOLVADEX; and raloxifene hydrochloride as EVISTA. Fulvestrant can be formulated as disclosed in U.S. Pat. No. 4,659,516 and is marketed as FASLODEX. A combination of the invention comprising a chemotherapeutic agent which is an anti-estrogen is particularly useful for the treatment of estrogen receptor positive tumors, e.g., breast tumors.

The term “anti-androgen”, as used herein, relates to any substance which is capable of inhibiting the biological effects of androgenic hormones and includes, but is not limited to, bicalutamide (CASODEX), which can be formulated, e.g., as disclosed in U.S. Pat. No. 4,636,505.

The term “gonadorelin agonist”, as used herein, includes, but is not limited to, abarelix, goserelin and goserelin acetate. Goserelin is disclosed in U.S. Pat. No. 4,100,274 and is marketed as ZOLADEX. Abarelix can be formulated, e.g., as disclosed in U.S. Pat. No. 5,843,901.

The term “topoisomerase I inhibitor”, as used herein, includes, but is not limited to, topotecan, irinotecan, gimatecan, camptothecin and its analogues, 9-nitrocamptothecin and the macromolecular camptothecin conjugate PNU-166148 (compound A1 in WO 99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The term “topoisomerase II inhibitor”, as used herein, includes, but is not limited to, the anthracyclines, such as doxorubicin, including liposomal formulation, e.g., CAELYX, daunorubicin, epirubicin, idarubicin and nemorubicin; the anthraquinones mitoxantrone and losoxantrone; and the podophillotoxines etoposide and teniposide. Etoposide is marketed as ETOPOPHOS; teniposide as VM 26-BRISTOL; doxorubicin as ADRIBLASTIN or ADRIAMYCIN; epirubicin as FARMORUBICIN; idarubicin as ZAVEDOS; and mitoxantrone as NOVANTRON.

The term “microtubule active agent” relates to microtubule stabilizing, microtubule destabilizing agents and microtublin polymerization inhibitors including, but not limited to, taxanes, e.g., paclitaxel and docetaxel; vinca alkaloids, e.g., vinblastine, especially vinblastine sulfate; vincristine, especially vincristine sulfate and vinorelbine; discodermolides; colchicines; and epothilones and derivatives thereof, e.g., epothilone B or D or a derivative thereof. Paclitaxel may be administered, e.g., TAXOL; docetaxel as TAXOTERE; vinblastine sulfate as VINBLASTIN R.P.; and vincristine sulfate as FARMISTIN. Discodermolide can be obtained, e.g., as disclosed in U.S. Pat. No. 5,010,099. Also included are Epotholine derivatives which are disclosed in U.S. Pat. No. 6,194,181, WO 98/10121, WO 98/25929, WO 98/08849, WO 99/43653, WO 98/22461 and WO 00/31247. Especially preferred are Epotholine A and/or B.

The term “alkylating agent”, as used herein, includes, but is not limited to, cyclophosphamide, ifosfamide, melphalan, nitrosourea (BCNU or Gliadel), lomustine and temozolomide. Cyclophosphamide can be administered, e.g., in the form as it is marketed, e.g., under the trademark CYCLOSTIN; ifosfamide as HOLOXAN; and temozolomide as TEMODAL.

The term “anti-neoplastic anti-metabolite” includes, but is not limited to, 5-fluorouracil (5-FU); capecitabine; gemcitabine; DNA de-methylating agents, such as 5-azacytidine and decitabine; methotrexate; edatrexate; and folic acid antagonists such as pemetrexed. Capecitabine can be administered, e.g., in the form as it is marketed, e.g., under the trademark XELODA; and gemcitabine as GEMZAR. Also included is the monoclonal antibody trastuzumab which can be administered, e.g., in the form as it is marketed, e.g., HERCEPTIN.

The term “platin compound”, as used herein, includes, but is not limited to, carboplatin, cis-platin, cisplatinum and oxaliplatin. Carboplatin can be administered, e.g., in the form as it is marketed, e.g., CARBOPLAT; and oxaliplatin as ELOXATIN.

The term “compound targeting/decreasing a protein or lipid kinase activity”, as used herein, includes, but is not limited to: protein tyrosine kinase and/or serine and/or threonine kinase inhibitors or lipid kinase inhibitors, e.g.,
i) compounds targeting, decreasing or inhibiting the activity of the platelet-derived growth factor-receptors (PDGFR), especially compounds which inhibit the PDGF receptor, e.g., a N-phenyl-2-pyrimidine-amine derivative, e.g., imatinib, SU101, SU6668 and GFB-111;
ii) compounds targeting, decreasing or inhibiting the activity of the fibroblast growth factor-receptors (FGFR);
iii) compounds targeting, decreasing or inhibiting the activity of the insulin-like growth factor I receptor (IGF-IR), especially compounds which inhibit the IGF-IR, such as those compounds disclosed in WO 02/092599, in particular trans-5-(3-benzyloxy-phenyl)-7-(3-pyrrolidin-1-ylmethyl-cyclobutyl)-7H-pyrrolo[2,3-d]pyrimidin-4-ylamine and cis-7-(3-azetidin-1-ylmethyl-cyclobutyl)-5-(3-benzyloxy-phenyl)-7H-pyrrolo[2,3-d]pyrimidin-4-ylamine or pharmaceutically acceptable salts of these compounds;
iv) compounds targeting, decreasing or inhibiting the activity of the Trk receptor tyrosine kinase family;
v) compounds targeting, decreasing or inhibiting the activity of the AxI receptor tyrosine kinase family;
vi) compounds targeting, decreasing or inhibiting the activity of the RET receptor tyrosine kinase;
vii) compounds targeting, decreasing or inhibiting the activity of the c-kit receptor tyrosine kinases, especially compounds which inhibit the c-Kit receptor, e.g., imatinib;
viii) compounds targeting, decreasing or inhibiting the activity of members of the c-Abl family and their gene-fusion products, e.g., Bcr-Abl kinase, such as especially compounds which inhibit the activity of c-Abl family members and their gene fusion products, e.g., a N-phenyl-2-pyrimidine-amine derivative, e.g., imatinib, PD180970, AG957, NSC 680410 or PD173955 from ParkeDavis;
ix) compounds targeting, decreasing or inhibiting the activity of members of the protein kinase C (PKC) and Raf family of serine/threonine kinases, members of the MEK, SRC, JAK, FAK, PDK and Ras/MAPK family members, or PI3 kinase (PI3K) family, or of the PI3-kinase-related kinase family, and/or members of the cyclin-dependent kinase family (CDK) and are especially those staurosporine derivatives disclosed in U.S. Pat. No. 5,093,330, e.g., midostaurin; examples of further compounds include, e.g., UCN-01; safingol; BAY 43-9006; Bryostatin 1; Perifosine; Ilmofosine; RO 318220 and RO 320432; GO 6976; Isis 3521; LY333531/LY379196; isochinoline compounds, such as those disclosed in WO 00/09495; FTIs; PD184352 or QAN697 (a PI3K inhibitor);
x) compounds targeting, decreasing or inhibiting the activity of protein tyrosine kinase inhibitors include imatinib mesylate (GLEEVEC/GLIVEC) or a tyrphostin. A tyrphostin is preferably a low molecular weight (Mᵣ<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556 and AG957 and adaphostin (4-{[(2,5-dihydroxyphenyl)methyl]amino}-benzoic acid adamantyl ester, NSC 680410); and
xi) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g., EGF receptor, ErbB2, ErbB3 and ErbB4 or bind to EGF or EGF-related ligands, and are in particular those compounds, proteins or antibodies generically and specifically disclosed in WO 97/02266, e.g., the compound of Example 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347, e.g., compound known as CP 358774, WO 96/33980, e.g., compound ZD 1839; and WO 95/03283, e.g., compound ZM105180, e.g., trastuzumab (Herceptin®), cetuximab, gefitinib (Iressa), erlotinib (Tarceva™), CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541.

By the term “antibody” is meant for example intact monoclonal antibodies, polyclonal antibodies, multi-specific antibodies formed from at least 2 intact antibodies, and antibody fragments so long as they exhibit the desired biological activity.

Compounds which target/decrease the activity of a protein or lipid phosphatase are, e.g., inhibitors of phosphatase 1, phosphatase 2A, PTEN or CDC25, e.g., okadaic acid or a derivative thereof.

Further anti-angiogenic compounds include compounds having another mechanism for their activity, e.g., unrelated to protein or lipid kinase inhibition, e.g., thalidomide (THALOMID) and TNP470.

Compounds which induce cell differentiation processes are e.g. retinoic acid, α-, γ- or δ-tocopherol or α-, γ- or δ-tocotrienol.

The term “cyclooxygenase inhibitor” as used herein includes, but is not limited to, e.g., Cox-2 inhibitors, 5-alkyl substituted 2-arylaminophenylacetic acid and derivatives, such as celecoxib (CELEBREX), rofecoxib (VIOXX), etoricoxib, valdecoxib (BEXTRA) or a 5-alkyl-2-arylaminophenylacetic acid, e.g., 5-methyl-2-(2′-chloro-6′-fluoroanilino)phenyl acetic acid (lumiracoxib, PREXIGE).

The term “bisphosphonate”, as used herein, includes, but is not limited to, etridonic, clodronic, tiludronic, pamidronic, alendronic, ibandronic, risedronic and zoledronic acid. Etridonic acid can be administered, e.g., in the form as it is marketed, e.g., DIDRONEL; clodronic acid as BONEFOS; tiludronic acid as SKELID; pamidronic acid as AREDIA; alendronic acid as FOSAMAX; ibandronic acid as BONDRANAT; risedronic acid as ACTONEL; and zoledronic acid as ZOMETA.

The term “heparanase inhibitor”, as used herein, refers to compounds which target, decrease or inhibit heparin sulphate degradation. The term includes, but is not limited to, PI-88.

The term “biological response modifier”, as used herein, refers to a lymphokine or interferons, e.g., interferon γ.

The term “inhibitor of Ras oncogenic isoforms”, e.g., H-Ras, K-Ras or N-Ras, as used herein, refers to compounds which target, decrease or inhibit the oncogenic activity of Ras, e.g., a farnesyl transferase inhibitors (FTIs), e.g., L-744832, DK8G557 or R115777 (Zamestra).

The term “telomerase inhibitor”, as used herein, refers to compounds which target, decrease or inhibit the activity of telomerase. Compounds which target, decrease or inhibit the activity of telomerase are especially compounds which inhibit the telomerase receptor, e.g., telomestatin.

The term “methionine aminopeptidase inhibitor”, as used herein, refers to compounds which target, decrease or inhibit the activity of methionine aminopeptidase. Compounds which target, decrease or inhibit the activity of methionine aminopeptidase are, e.g., bengamide or a derivative thereof.

The term “proteasome inhibitor”, as used herein, refers to compounds which target, decrease or inhibit the activity of the proteasome. Compounds which target, decrease or inhibit the activity of the proteasome include, e.g., PS-341 and MLN 341.

The term “matrix metalloproteinase inhibitor” or “MMP inhibitor”, as used herein, includes, but is not limited to, collagen peptidomimetic and non-peptdomimetic inhibitors; tetracycline derivatives, e.g., hydroxamate peptidomimetic inhibitor batimastat; and its orally-bioavailable analogue marimastat (BB-2516), prinomastat (AG3340), metastat (NSC 683551) BMS-279251, BAY 12-9566, TAA211, MMI270B or AAJ996.

The term “agents used in the treatment of hematologic malignancies”, as used herein, includes, but is not limited to, FMS-like tyrosine kinase inhibitors, e.g., compounds targeting, decreasing or inhibiting the activity of Flt-3; interferons; cytosine arabinoside (Ara-C); bisulfan; and ALK inhibitors, i.e. compounds which target, decrease or especially inhibit anaplastic lymphoma kinase (ALK).

The term “FMS-like tyrosine kinase inhibitors”, as used herein, includes, but is not limited to, compounds which target, decrease or inhibit the activity of FMS-like tyrosine kinase receptors such as especially compounds, proteins or antibodies which inhibit Flt-3, e.g., PKC412, midostaurin, a staurosporine derivative, SU11248 and MLN518.

The term “HSP90 inhibitors”, as used herein, includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17-AAG), a geldanamycin derivative; other geldanamycin-related compounds; radicicol and HDAC inhibitors.

The term “histone deacetylase inhibitors” or “HDAC inhibitors” relates to compounds which target, decrease or especially inhibit the activity of histone deacetylase (HDAC), such as sodium butyrate and suberoylanilide hydroxamic acid (SAHA). Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl}-2E-2-propenamide, or a pharmaceutically acceptable salt thereof, especially the lactate salt.

The term “mTOR inhibitors” relates to compounds which target, decrease or inhibit the activity/function of the serine/threonine mTOR kinase family and are especially compounds, proteins or antibodies which inhibit members of the mTOR kinase family, e.g., CCI-779, ABT578, SAR543, rapamycin and derivatives/analogs thereof, AP23573 and AP23841 from Ariad, everolimus (CERTICAN, RAD001) and sirolimus (RAPAMUNE).

“Somatostatin receptor antagonists”, as used herein, refers to agents which target, treat or inhibit the somatostatin receptor, such as octreotide and SOM230.

The term “integrin antagonists”, as used herein, includes, but is not limited to, e.g. ανβ3 antagonists and ανβ5 antagonists.

“Tumor cell damaging approaches” refers to approaches, such as ionizing radiation. The term “ionizing radiation”, referred to above and hereinafter, means ionizing radiation that occurs as either electromagnetic rays, such as X-rays and gamma rays; or particles, such as alpha and beta particles. Ionizing radiation is provided in, but not limited to, radiation therapy and is known in the art. See Hellman, Cancer, 4^{th} Edition, Vol. 1, Devita et al., Eds., pp. 248-275 (1993).

The term “anti-leukemic compounds” includes, e.g., Ara-C, a pyrimidine analog, which is the 2′-α-hydroxy ribose (arabinoside) derivative of deoxycytidine. Also included is the purine analog of hypoxanthine, 6-mercaptopurine (6-MP) and fludarabine phosphate.

The term “EDG binders” as used herein refers to a class of immunosuppressants that modulates lymphocyte recirculation, such as FTY720.

The term “ribonucleotide reductase inhibitors” refers to pyrimidine or purine nucleoside analogs including, but not limited to, fludarabine and/or Ara-C; 6-thioguanine; 5-FU; cladribine; 6-mercaptopurine, especially in combination with Ara-C against ALL; and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8. See Nandy et al., Acta Oncologica, Vol. 33, No. 8, pp. 953-961 (1994).

The term “S-adenosylmethionine decarboxylase inhibitors”, as used herein, includes, but is not limited to, the compounds disclosed in U.S. Pat. No. 5,461,076.

Antibodies against VEGF or VEGFR are especially antibodies, in particular monoclonal antibodies, which inhibit the activity of VEGF and/or VEGFR and includes, but is not limited to, bevacizumab (AVASTIN), HuMV833, IMC-1C11 and ranibizumab (RhuFab).

“Photodynamic therapy”, as used herein, refers to a therapy which uses certain chemicals known as photosensitizing agents which are activated by a laser. Examples of photodynamic therapy includes treatment with agents, such as, e.g., verteporfin (VISUDYNE, BPD-MA) and porfimer sodium.

“Angiostatic steroids”, as used herein, refers to agents which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to agents, such as, e.g., fluocinolone and dexamethasone.

AT1 receptor antagonists include agents, such as valsartan (DIOVAN).

ACE inhibitors include benazepril (CIBACEN), enazepril (LOTENSIN), captopril, enalapril, fosinopril, lisinopril, moexipril, quinapril, ramipril, perindopril and trandolapril.

Other chemotherapeutic agents include, but are not limited to, plant alkaloids, hormonal agents and antagonists, biological response modifiers, preferably lymphokines or interferons, antisense oligonucleotides or oligonucleotide derivatives; or miscellaneous agents or agents with other or unknown mechanism of action.

Comprised are likewise the corresponding stereoisomers, as well as the corresponding crystal modifications, e.g., solvates and polymorphs, of the active ingredients of the combinations disclosed herein. The compounds used as active ingredients in the combinations disclosed herein can be prepared and administered as described in the cited documents, respectively.

The structure of the active agents identified by code numbers, generic or trade names may be taken from the actual edition of the standard compendium “The Merck Index” or from databases, e.g., Patents International, e.g., IMS World Publications, or the publications mentioned above and below. The corresponding content thereof is hereby incorporated by reference.

It will be understood that references to the components (a) and (b) are meant to also include the pharmaceutically acceptable salts of any of the active substances. If active substances comprised by components (a) and/or (b) have, for example, at least one basic center, they can form acid addition salts. Corresponding acid addition salts can also be formed having, if desired, an additionally present basic center. Active substances having an acid group, e.g., COOH, can form salts with bases. The active substances comprised in components (a) and/or (b) or a pharmaceutically acceptable salts thereof may also be used in form of a hydrate or include other solvents used for crystallization.

### The VEGF Inhibitor Compounds

VEGF inhibitors are especially compounds, proteins or antibodies which inhibit at least one VEGF receptor tyrosine kinase.

VEGF inhibitors for use in the present invention include those of formula (I)

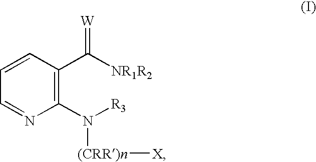

wherein for formula (I) the variables are:
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
   or of a N-oxide or a possible tautomer thereof;
   or of a pharmaceutically acceptable salt.

The general terms used hereinbefore and hereinafter preferably have within the context of this disclosure for formula (I) the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms, e.g., in compounds of formula (I), wherein R or R′ is lower alkyl, may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula (I).

X is preferably pyridyl or phenyl, most preferred it is 3- or 4-pyridyl.

In a preferred embodiment of the invention, X is substituted by lower alkoxy.

In further a very preferred embodiment of the invention, X has the substructure X′

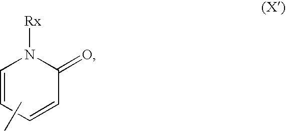

wherein Rx is hydrogen or lower alkyl.

R₂ is preferably phenyl which is mono- or disubstituted by lower alkyl, lower alkynyl, halogen, preferably fluoro, and trifluoromethyl; or cycloalkyl, preferably cyclohexyl substituted by lower alkyl, preferably tert-butyl.

R₃ is preferably hydrogen. W is preferably O. The integer n is preferably 1 or 2, very preferably 1.

Lower alkyl is preferably alkyl with from and including 1 up to and including 7, preferably from and including 1 to and including 5, and is linear or branched; preferably, lower alkyl is pentyl, such as n-pentyl, butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or acetyl.

“Aryl” is an aromatic radical which is bound to the molecule via a bond located at an aromatic ring carbon atom of the radical. In a preferred embodiment, aryl is an aromatic radical having 6-14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or di-substituted amino, halogen, lower alkyl, substituted alkyl, lower alkenyl, lower alkynyl, lower alkanoyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenyl, phenoxy, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy. Aryl is more preferably phenyl or naphthyl, which in each case is either unsubstituted or independently substituted by one or two substituents selected from the group comprising halogen, especially fluorine, chlorine or bromine; hydroxy; hydroxy, etherified by lower alkyl, e.g., methyl or by halogen-lower alkyl, e.g., trifluoromethyl; lower alkyl, e.g., methyl or propyl; lower alkynyl, such as 1-propynyl; esterified carboxy, especially lower alkoxy carbonyl, e.g., methoxy carbonyl, n-propoxy carbonyl or iso-propoxy carbonyl; N-mono-substituted carbamoyl, in particular carbamoyl monosubstituted by lower alkyl, e.g., methyl, n-propyl or iso-propyl; substituted alkyl, especially lower alkyl, e.g., methyl or ethyl, substituted by lower alkoxy carbonyl, e.g., methoxy carbonyl or ethoxy carbonyl; and halogen-lower alkyl, most preferably trifluoromethyl.

Aryl in the form of phenyl which is substituted by lower alkylene dioxy bound to two adjacent C-atoms, such as methylenedioxy, is preferably 3,4-methylenedioxyphenyl.

A cycloalkyl group is preferably cyclopentyl, cyclohexyl or cycloheptyl, and may be unsubstituted or substituted by one or more, especially one or two, substituents selected from the group defined above as substituents for aryl, most preferably by lower alkyl, such as methyl; lower alkoxy, such as methoxy or ethoxy; or hydroxy.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-1-lower alkylamino, N-lower alkanoylamino, hydroxy, cyano, carboxy, lower alkoxycarbonyl and phenyl-lower alkoxycarbonyl. Trifluoromethyl is especially preferred.

Mono- or di-substituted amino is especially amino substituted by one or two radicals selected independently of one another from lower alkyl, such as methyl; hydroxy-lower alkyl, such as 2-hydroxyethyl; phenyl-lower alkyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl and carbamoyl; and is preferably N-lower alkylamino, such as N-methylamino, hydroxy-lower alkylamino, such as 2-hydroxyethylamino, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino.

Halogen is especially fluorine, chlorine, bromine, or iodine, especially fluorine, chlorine or bromine.

Etherified hydroxy is especially C₈₋₂₀alkyloxy, such as n-decyloxy, lower alkoxy (preferred), such as methoxy, ethoxy, isopropyloxy or n-pentyloxy, phenyl-lower alkoxy, such as benzyloxy, or also phenyloxy, or as an alternative or in addition to the previous group C₈₋₂₀alkyloxy, such as n-decyloxy, halogen-lower alkoxy, such as trifluoromethyloxy or 1,1,2,2-tetrafluoroethoxy.

Esterified hydroxy is especially lower alkanoyloxy, benzoyloxy, lower alkoxycarbonyloxy, such as tert-butoxycarbonyloxy or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl or phenyloxycarbonyl.

Alkanoyl is primarily alkylcarbonyl, especially lower alkanoyl, e.g., acetyl.

N-Mono- or N,N-disubstituted carbamoyl is especially substituted by one or two substituents independently selected from lower alkyl, phenyl-lower alkyl, and hydroxy-lower alkyl, at the terminal nitrogen atom.

Alkylphenylthio is especially lower alkylphenylthio.

Alkylphenylsulfonyl is especially lower alkylphenylsulfonyl.

Alkylphenylsulfinyl is especially lower alkylphenylsulfinyl.

A mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted refers to a heterocyclic moiety that is unsaturated in the ring binding the heteroaryl radical to the rest of the molecule in formula (I) and is preferably a ring, where at least in the binding ring, but optionally also in any annealed ring, one or more, preferably 14, most preferably 1 or 2, carbon atoms are replaced each by a heteroatom selected from the group consisting of nitrogen, oxygen and sulfur; where the binding ring preferably has 5-12, more preferably 5-7 ring atoms; and may be unsubstituted or substituted by one or more, especially one or two, substituents selected from the group defined above as substituents for aryl, most preferably by lower alkyl, such as methyl; lower alkoxy, such as methoxy or ethoxy; or hydroxy; preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl and benzo[d]pyrazol. More preferably the mono- or bicyclic heteroaryl group is selected from the group consisting of pyrrolyl, benzimidazolyl, such as 1-benzimidazolyl, indazolyl, especially 5-indazolyl; pyridyl, especially 2-, 3- or 4-pyridyl; isoquinolinyl, especially 3-isoquinolinyl; quinolinyl, especially 4-quinolinyl; indolyl, especially 3-indolyl, thiazolyl or benzo[d]pyrazol. In one preferred embodiment of the invention the pyridyl radical is substituted by hydroxy in ortho position to the nitrogen atom and hence exists at least partially in the form of the corresponding tautomer which is pyridin-(1H)2-one.

Heterocyclyl is especially a five or six-membered heterocyclic system with 1 or 2 heteroatoms selected from the group comprising nitrogen, oxygen and sulfur, which may be unsaturated or wholly or partly saturated, and is unsubstituted or substituted especially by lower alkyl, such as methyl; a radical selected from 2-methylpyrimidin-4-yl, oxazol-5-yl, 2-methyl-1,3-dioxolan-2-yl, 1H-pyrazol-3-yl and 1-methyl-pyrazol-3-yl is preferred.

Salts are especially the pharmaceutically acceptable salts of compounds of formula (I).

Such salts are formed, e.g., as acid addition salts, preferably with organic or inorganic acids, from compounds of formula (I) with a basic nitrogen atom, especially the pharmaceutically acceptable salts. Suitable inorganic acids are, e.g., halogen acids, such as hydrochloric acid, sulfuric acid or phosphoric acid. Suitable organic acids are, e.g., carboxylic, phosphonic, sulfonic or sulfamic acids, e.g., acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid or citric acid; amino acids, such as glutamic acid or aspartic acid; maleic acid; hydroxylmaleic acid; methylmaleic acid; cyclohexanecarboxylic acid; adamantanecarboxylic acid; benzoic acid; salicylic acid; 4-aminosalicylic acid; phthalic acid; phenylacetic acid; mandelic acid; cinnamic acid; methane- or ethane-sulfonic acid; 2-hydroxyethanesulfonic acid; ethane-1,2-disulfonic acid; benzenesulfonic acid; 2-naphthalenesulfonic acid; 1,5-naphthalene-disulfonic acid; 2-, 3- or 4-methylbenzenesulfonic acid; methylsulfuric acid; ethylsulfuric acid; dodecylsulfuric acid; N-cyclohexylsulfamic acid; N-methyl-; N-ethyl-; or N-propyl-sulfamic acid or other organic protonic acids, such as ascorbic acid.

High preference is given to a compound selected from the group consisting of:
2-[2-(4-Pyridyl)ethyl]amino-N-[3-(trifluoromethyl)phenyl-3-pyridinecarboxamide;
2-[(4-Pyridyl)methyl]amino-N-[3-(trifluoromethyl)phenyl]-3-pyrdinecarboxamide;
2-[(2-Methyl-4-pyridyl)methyl]amino-N-[3-(trifluoromethyl)phenyl]-3-pyridinecarboxamide;
2-[(6-Methoxy-3-pyridyl)methyl]amino-N-[3-(trifluoromethyl)phenyl]-3-pyridinecarboxamide;
2-[(4-Pyridyl)methyl]amino-N-[3,4-bis(trifluoromethyl)-phenyl]-3-pyridinecarboxamide;
2-[(4-Pyridyl)methyl]amino-N-[5-fluoro-3-trifluoromethyl-phenyl]-3-pyrdinecarboxamide;
2-[(4-Pyridyl)methyl]amino-N-(trans-4-tert-butyl-cyclohexane)-3-pyrdinecarboxamide;
2-[(4-Pyridyl)methyl]amino-N-(4n-propyl-phenyl)-3-pyridinecarboxamide;
2-[(4-Pyridyl)methyl]amino-N-(4-n-butyl-phenyl)-3-pyridinecarboxamide;
2-[(4-Pyridyl)methyl]amino-N-(4-n-pentyl-phenyl)-3-pyridinecarboxamide;
2-[(4-Pyridyl)methyl]amino-N-[4-(1-propynyl)-phenyl]-3-pyridinecarboxamide;
2-[(4-Pyridyl)methyl]amino-N-(5-indazolyl)-3-pyridinecarboxamide;
2-[(4-Pyridyl)methyl]amino-N-(3-isoquinolinyl)-3-pyridinecarboxamide;
2-[(Pyridin-6(1H)-on-3-yl)methyl]amino-N-[3-(trifluoromethyl)phenyl]-3-pyridinecarboxamide; and
   the pharmaceutically acceptable salt thereof.

Furthermore, high preference is given to a compound selected from the group of compounds consisting of:
2-(Phenylmethylamino-N-[3-(trifluoromethyl)phenyl]-3-pyridine-carboxamide, hydrochloride;
2-[(4-Pyridyl)methylamino]-N-[2-fluoro-3-(trifluoromethyl)phenyl]-3-pyrdinecarboxamide;
2-[(4-Pyridyl)methylamino]-N-[4-bromo-3-(trifluoromethyl)phenyl]-3-pyridinecarboxamide;
2-[(4-Pyridyl)methylamino]-N-[2-methyl-3-(trifluoromethyl)phenyl]-3-pyridinecarboxamide;
2-[(4-Pyridyl)methylamino]-N-[2-methyl-5-(trifluoromethyl)phenyl]-3-pyridinecarboxamide;
2-[(4-Pyridyl)methylamino]-N-(cis-4-tert-butyl-cyclohexyl)-3-pyridinecarboxamide;
2-[(6-Methoxypyrid-3-yl)methylamino]-N-[4-bromo-3-(trifluoromethyl)phenyl]-3-pyridinecarboxamide;
2-[(6-Methoxypyrid-3-yl)methylamino]-N-[2-fluoro-3-(trifluoromethyl)phenyl]-3-pyridinecarboxamide;
2-[(6-Methoxypyrid-3-yl)methylamino]-N-[2-methyl-3-(trifluoromethyl)phenyl]-3-pyridinecarboxamide;
2-[(1-Oxido-4-pyridyl)methylamino]-N-[3-(trifluoromethyl)phenyl]-3-pyridinecarboxamide;
2-[3-(N-methyl-carboxamido)phenyl]methylamino]-N-[3-(trifluoromethyl)phenyl]-3-pyridinecarboxamide;
2-[(1-Methyl-pyridin-2(1H)-on-5-yl)methylamino]-N-[3-(trifluoromethyl)phenyl]-3-pyridine-carboxamide;
2-[(6-Methoxypyrid-3-yl)methylamino]-N-[4-propynyl-3-(trifluoromethyl)phenyl]-3-pyridinecarboxamide;
2-[(4-Pyridyl)methylamino]-N-[4-propynyl-3-(trifluoromethyl)phenyl]-3-pyridinecarboxamide;
2-[(Pyridin-2(1-on-5-yl)methyl]amino-N-[4-propynyl-3-(trifluoromethyl)phenyl]-3-pyridinecarboxamide;
2-[(Pyridin-2(1H)-on-5-yl)methyl]amino-N-[3-(trifluoromethyl)phenyl]-3-pyrdinecarboxamide;
2-[(3-Hydroxyphenyl)methyl]amino-N-[3-(trifluoromethyl)phenyl]-3-pyridinecarboxamide;
2-[(Pyridin-2(1-on-5-yl)methyl]amino-N-[4-bromo-3-(trifluoromethyl)phenyl]-3-pyridinecarboxamide;
2-[(Pyridin-2(1H)-on-5-yl)methyl]amino-N-[2-fluoro-3-(trifluoromethyl)phenyl]-3-pyridinecarboxamide;
2-[(Pyridin-2(1]-on-5-yl)methyl]amino-N-[2-methyl-3-(trifluoromethyl)phenyl]-3-pyridinecarboxamide;
2-[(Pyridin-2(1H)-on-5-yl)methyl]amino-N-[4-propyl-3-(trifluoromethyl)phenyl]-3-pyridinecarboxamide;
2-[(6-Methoxypyrid-3-yl)methylamino]-N-[4-propyl-3-(trifluoromethyl)phenyl]-3-pyridinecarboxamide;
2-[(4-Pyridyl)methyl]amino-N-[4-(n-propyl)-3-(trifluoromethyl)phenyl]-3-pyridinecarboxamide;
2-[(4-Pyridyl)methyl]amino-N-(5-thiazolyl)-3-pyridinecarboxamide;
2-[(4-Hydroxyphenyl)methyl]amino-N-[3-(trifluoromethyl)phenyl]-3-pyridine-carboxamide;
2-[(4-Pyridyl)methyl]amino-N-(benzo[d]pyrazol-5-yl)-3-pyridinecarboxamide;
2-[(6-Methoxy-3-pyridyl)methyl]amino-N-(3-isoquinolinyl)-3-pyridinecarboxamide;
2-[(6-Methoxy-3-pyridyl)methyl]amino-N-(benzo[d]pyrazol-5-yl)-3-pyridinecarboxamide;
2-[(Pyridin-2(1H)-on-5-yl)methyl]amino-N-(3-isoquinolinyl)-3-pyridinecarboxamide;
2-[(Pyridin-2(1H)-on-5-yl)methyl]amino-N-(benzo[d]pyrazol-5-yl)-3-pyridinecarboxamide;
2-[(Pyridin-2(1H)-on-5-yl)methyl]amino-N-(cis-4-tert-butyl-cyclohexyl)-3-pyridinecarboxamide;
2-[(Pyridin-2(1H)-on-5-yl)methyl]amino-N-(trans-4-tert-butyl-cyclohexyl)-3-pyridinecarboxamide;
2-[(1-Oxido-4-pyridyl)methylamino]-N-[4-propyl-3-(trifluoromethyl)phenyl]-3-pyridinecarboxamide;
2-[(Pyridin-2(1H)-on-5-yl)methyl]amino-N-[4-ethyl-3-(trifluoromethyl)phenyl]-3-pyridinecarboxamide;
2-[(Pyridin-2(1-on-5-yl)methyl]amino-N-[3,4-bis(trifluoromethyl)phenyl]-3-pyridinecarboxamide; and
2-[(1-Methyl-pyridin-2(1H) on-5-yl)methylamino]-N-[3,4-bis(trifluoromethyl)phenyl]-3-pyridinecarboxamide; and
   the pharmaceutically acceptable salts thereof.

The most preferred compound of formula (I) is 2-[(pyridin-6(1H)-on-3-yl)methyl]amino-N-[3-(trifluoromethyl)phenyl]-3-pyridine-carboxamide or a pharmaceutically acceptable salt thereof.

VEGF inhibitors of formula (I) and their preparation are disclosed in WO 01/55114, published Aug. 2, 2001, and are herewith incorporated.

Other VEGF inhibitors include compounds of formula (II)

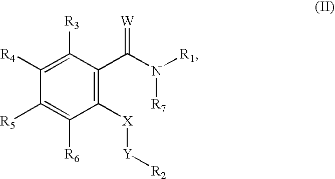

wherein for formula (II) the variables are:
W is O or S;
X is NR₈;
Y is CR₉R₁₀—(CH₂)ₙ,
wherein
   R₉ and R₁₀ are, independently, of each other hydrogen or lower alkyl; and
   n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y=SO₂ cannot represent 2,1,3-benzothiadiazol-4-yl;
any of R₃, R₄, R₅ and R₆, independently of the other, is H or a substituent other than hydrogen; and
R₇ and R₈, independently of each other, are H or lower alkyl;
   or of an N-oxide;
   or a pharmaceutically acceptable salt thereof.

The general terms used hereinbefore and hereinafter preferably have, within the context of formula (II) the following meanings, unless otherwise indicated.

The prefix “lower” denotes a radical having up to and including a maximum of 7, especially up to and including a maximum of 4 carbon atoms, the radicals in question being either linear or branched with single or multiple branching.

Where the plural form is used for compounds, salts and the like, this is taken to mean also a single compound, salt or the like.

Any asymmetric carbon atoms, e.g., in compounds of formula (II), wherein R₉ is lower alkyl) may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula (II).

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

The index n is preferably 0 or 1, especially 0.

Y is preferably methylene (CH₂) or ethylene (CH₂—CH₂), most preferably methylene.

“Aryl” is an aromatic radical which is bound to the molecule via a bond located at an aromatic ring carbon atom of the radical. In a preferred embodiment, aryl is an aromatic radical having 6-14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, alkyl, substituted alkyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenyl, phenoxy, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, lower alkenyl, lower alkanoyl, halogen-lower alkylmercapto, halogen-lower alkylsulfonyl, such as, especially trifluoromethane sulfonyl, dihydroxybora (—B(OH)₂), heterocyclyl and lower alkylene dioxy bound at adjacent C-atoms of the ring, such as methylene dioxy; aryl is preferably phenyl or naphthyl, which in each case is either unsubstituted or independently substituted by one or two substituents selected from the group comprising halogen, especially fluorine, chlorine or bromine; hydroxy; hydroxy, etherified by lower alkyl, e.g., methyl, or by halogen-lower alkyl, e.g., trifluoromethyl; esterified carboxy, especially lower alkoxy carbonyl, e.g., methoxy carbonyl, n-propoxy carbonyl or iso-propoxy carbonyl; N-mono-substituted carbamoyl, in particular, carbamoyl monosubstituted by lower alkyl, e.g., methyl, n-propyl or isopropyl; lower alkyl, especially methyl, ethyl or propyl; substituted alkyl, especially lower alkyl, e.g., methyl or ethyl, substituted by lower alkoxy carbonyl, e.g., methoxy carbonyl or ethoxy carbonyl; halogen-lower alkyl, especially trifluoromethyl; lower alkylsulfinyl, such as methylsulfinyl, and lower alkanesulfonyl, such as methane sulfonyl. Aryl is preferably 3- or 4-chlorophenyl, 3-bromophenyl, 4-phenoxyphenyl, 2,3- or 4-methylphenyl, 4-methoxyphenyl, 3- or 4-tert-butylphenyl, 4-n-propylphenyl, 4-trifluoromethylphenyl, 3-trifluoromethylphenyl, 3-trifluoromethoxyphenyl, 3,4-(trifluoromethyl)phenyl, 3-fluoro-4-methylphenyl, 3-chloro-4-methylphenyl, 4-chloro-3-trifluoromethylphenyl, 3-chloro-5-trifluoromethylphenyl, 4-methylsulfinylphenyl, 4-methanesulfonylphenyl, 4-biphenyl, naphthyl, 2-naphthyl; tetrahydronaphthyl, in particular, 5,6,7,8-tetrahydronaphthyl; hydroxynaphthyl, in particular, 7-hydroxynaphthyl, 8-hydroxynaphthyl or 8-hydroxy-2-naphthyl; methoxynaphthyl, in particular, 4-methoxy-2-naphthyl; halonaphthyl, in particular, 4-chloronaphthyl or 3-bromo-2-naphthyl.

Mono- or di-substituted amino is especially amino substituted by one or two radicals selected independently of one another from lower alkyl, such as methyl; hydroxy-lower alkyl, such as 2-hydroxyethyl; phenyl-lower alkyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl and carbamoyl; and is preferably N-lower alkylamino, such as N-methylamino, hydroxy-lower alkylamino, such as 2-hydroxyethylamino, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino.

Halogen is especially fluorine, chlorine, bromine or iodine, especially fluorine, chlorine or bromine.

In the preferred embodiment, alkyl has up to a maximum of 12 carbon atoms and is especially lower alkyl, especially methyl, or also ethyl, n-propyl, isopropyl or tert-butyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl and phenyl-lower alkoxycarbonyl. Trifluoromethyl is especially preferred.

Etherified hydroxy is especially C₈₋₂₀alkyloxy, such as n-decyloxy, lower alkoxy (preferred), such as methoxy, ethoxy, isopropyloxy, or n-pentyloxy; phenyl-lower alkoxy, such as benzyloxy; or also phenyloxy, or as an alternative or in addition to the previous group C₈₋₂₀alkyloxy, such as n-decyloxy or halogen-lower alkoxy, such as trifluoromethyloxy or 1,1,2,2-tetrafluoroethoxy.

Esterified hydroxy is especially lower alkanoyloxy, benzoyloxy, lower alkoxycarbonyloxy, such as tert-butoxycarbonyloxy; or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl; phenyl-lower alkoxycarbonyl; or phenyloxycarbonyl.

Alkanoyl is primarily alkylcarbonyl, especially lower alkanoyl, e.g., acetyl.

N-mono- or N,N-disubstituted carbamoyl is especially substituted by one or two substituents independently selected from lower alkyl, phenyl-lower alkyl, and hydroxy-lower alkyl, at the terminal nitrogen atom.

Alkylphenylthio is especially lower alkylphenylthio.

Alkylphenylsulfonyl is especially lower alkylphenylsulfonyl.

Alkylphenylsulfinyl is especially lower alkylphenylsulfinyl.

Heterocyclyl is especially a 5 or 6-membered heterocyclic system with 1 or 2 heteroatoms selected from the group comprising nitrogen, oxygen and sulfur, which may be unsaturated or wholly or partly saturated, and is unsubstituted or substituted especially by lower alkyl, such as methyl; a radical selected from 2-methylpyrimidin-4-yl, oxazol-5-yl, 2-methyl-1,3-dioxolan-2-yl, 1H-pyrazol-3-yl and 1-methyl-pyrazol-3-yl is preferred.

Aryl in the form of phenyl which is substituted by lower alkylene dioxy bound to two adjacent C-atoms, such as methylenedioxy, is preferably 3,4-methylenedioxyphenyl.

Heteroaryl refers to a heterocyclic moiety that is unsaturated in the ring binding the heteroaryl radical to the rest of the molecule in formula (II) and is preferably mono-, bi- or tricyclic, preferably mono- or bicyclic; where at least in the binding ring, but optionally also in any annealed ring, one or more, preferably 1-4, most preferably 3 or 4 carbon atoms are replaced each by a heteroatom selected from the group consisting of nitrogen, oxygen and sulfur; where the binding ring preferably has 5-12, more preferably 5-7 ring atoms; and may be unsubstituted or substituted by one or more, especially one or two, substituents selected from the group defined above as substituents for aryl, most preferably by lower alkyl, such as methyl; preferably heteroaryl is selected from thienyl, furyl, pyranyl, thianthrenyl, isobenzofuranyl, benzofuranyl, chromenyl, 2H-pyrrolyl, pyrrolyl, lower-alkyl substituted imidazolyl, benzimidazolyl, pyrazolyl, thiazolyl, isothiazolyl, oxazolyl, isoxazolyl, pyridyl, pyrazinyl, pyrimidinyl, pyridazinyl, indolizinyl, isoindolyl, 3H-indolyl, indolyl, indazolyl, triazolyl, tetrazolyl, purinyl, 4H-quinolizinyl, isoquinolyl, quinolyl, phthalazinyl, naphthyridinyl, quinoxalyl, quinazolinyl, cinnolinyl, pteridinyl, carbazolyl, phenanthridinyl, acridinyl, perimidinyl, phenanthrolinyl and furazanyl; more preferably selected from the group consisting of triazolyl, especially 1,2,4-triazolyl, 1,2,3-triazolyl or 1,3,4-triazolyl; pyridyl, especially 2-, 3- or 4-pyridyl; indolyl, especially 3-indolyl; lower-alkylthiazolyl, especially 2-(4-methylthiazolyl); pyrrolyl, especially 1-pyrrolyl; lower alkylimidazolyl, especially 4-(1-methylimidazolyl), 4-(2-methylimidazolyl) or 4-(5-methylimidazolyl); benzimidazolyl, such as 1-benzimidazolyl; or tetrazolyl, such as 5-(1,2,3,4-tetrazolyl).

A mono- or bicyclic heteroaryl group comprising one or more ring nitrogen atoms is preferably a heteroaryl group as defined above for heteroaryl, with the proviso that preferably at least one nitrogen is present as ring heteroatom in the binding ring (that is, the ring from which the bond starts that binds the heteroaryl moiety to the rest of the molecule) and with the exception that R₂ cannot represent 2-phthalimidyl, and in case of Y═SO₂R₂ cannot represent 2,1,3-benzothiadiazol-4-yl. Preferred is imidazolyl, especially imidazol-4-yl; quinolyl, especially 3-, 4-, 5-quinolyl; naphthyridinyl, especially 3-(1,8-naphthyridinyl) or 4-(1,8-naphthyridinyl); or especially a moiety of the formula (IIb) or (IIc)

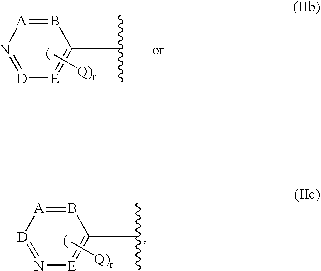

wherein
r is 0-2;
A, B, D and E are, independently of one another, N or CH, with the stipulation that not more than 2 of these radicals are N; preferably, each of A, B, D and E is CH; and
Q is lower alkyl, especially methyl, hydroxy, lower alkoxy, especially methoxy, lower thioalkyl, especially methylthio, or halogen, especially fluoro, chloro or bromo.

Very preferably R₂ is 3-pyridyl, 4-pyridyl, 4-quinolinyl or 5-quinolinyl. Most preferably, R₂ is 4-pyridyl.

A substituent other than hydrogen is preferably selected from amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, lower alkylthio, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, lower alkenyl, lower alkanoyl, halogen-lower alkylmercapto, halogen-lower alkylsulfonyl, such as especially trifluoromethane sulfonyl and heterocyclyl. Two substitutents other than hydrogen bound at adjacent C-atoms of the ring can also represent lower alkylene dioxy, such as methylene dioxy ethylene dioxy. Preferably, a substituent other than hydrogen is lower alkyl or halogen, especially methyl, chloro or fluoro.

Preferably, R₇ and R₈ are hydrogen, and R₃, R₄, R₅ and R₆ each are independently hydrogen, chloro or fluorine.

Salts are especially the pharmaceutically acceptable salts of compounds of formula (II).

Such salts are formed, e.g., as acid addition salts, preferably with organic or inorganic acids, from compounds of formula (II) with a basic nitrogen atom, especially the pharmaceutically acceptable salts. Suitable inorganic acids are, e.g., halogen acids, such as hydrochloric acid, sulfuric acid or phosphoric acid. Suitable organic acids are, e.g., carboxylic, phosphonic, sulfonic or sulfamic acids, e.g., acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

High preference is given to a compound selected from:
2-[(4-Pyridyl)methyl]amino-N-(4-trifluoromethylphenyl)benzamide;
2-[(4-Pyridyl)methyl]amino-N-(4-chlorophenyl)benzamide;
2-[(4-Pyridyl)methyl]amino-N-(4-methylphenyl)benzamide;
2-[(4-Pyridyl)methyl]amino-N-(3-fluoro-4-methylphenyl)benzamide;
2-[(4-Pyridyl)methyl]amino-N-(4-chloro-3-trifluoromethylphenyl)benzamide;
2-[(4-Pyridyl)methyl]amino-N-(3-chloro-5-trifluoromethylphenyl)benzamide;
2-[(4-Pyridyl)methyl]amino-N-(4-methylphenyl)-6-methylbenzamide; and
2-[(4-Quinolyl)methyl]amino-N-(4-chlorophenyl)benzamide;
   or a pharmaceutically acceptable salt thereof.

Furthermore, high preference is given to a compound selected from:
2-[(4-Pyridyl)methyl]amino-N-[3-fluoro-(4-trifluoromethyl)phenyl]benzamide;
2-[(4-Pyridyl)methyl]amino-N-phenylbenzamide;
2-[(4-Pyridyl)methyl]amino-N-[4-fluoro-3-(trifluoromethyl)phenyl]benzamide;
2-[(4-Pyridyl)methyl]amino-N-[3-fluoro-5-(trifluoromethyl)phenyl]benzamide;
2-[(4-Pyridyl)methyl]amino-N-[3,5-(bistrifluoromethyl)phenyl]benzamide;
2-[(4-Pyridyl)methyl]amino-N-[3,4-bis-(trifluoromethyl)phenyl]benzamide;
2-[(4-Pyridyl)methyl]amino-N-[3-methoxy-5-(trifluoromethyl)phenyl]benzamide;
2-[(4-Pyridyl)methyl]amino-N-[3-(trifluoromethyl)phenyl]benzamide;
2-[(4-Pyridyl)methyl]amino-N-[3-(1,1-dimethylethyl)phenyl]benzamide;
2-[(4-Pyridyl)methyl]amino-N-(3-cyanophenyl)benzamide;
2-[(4-Pyridyl)methyl]amino-N-[(3-methylthio)phenyl]benzamide;
2-[(4-Pyridyl)methyl]amino-N-(3-acetylaminophenyl)benzamide;
2-[(4-Pyridyl)methyl]amino-N-[3-[(aminocarbonyl)amino]phenyl]benzamide;
2-[(4-Pyridyl)methyl]amino-N-[3-(dimethylamino)phenyl]benzamide;
5-Methoxy-2-[(4-pyridyl)methyl]amino-N-[3-(trifluoromethyl)phenyl]benzamide;
3-Methyl-2-[(4-pyridyl)methyl]amino-N-[3-(trifluoromethyl)phenyl]benzamide;
4,5-Difluoro-2-[(4-pyridyl)methyl]amino-N-[3-(trifluoromethyl)phenyl]benzamide;
2-[(4-Pyridyl)methyl]amino-N′-methyl-N′-[3-(trifluoromethyl)phenyl]benzamide;
2-[(4-Pyridyl)methyl]amino-N-[(3-methylsulphonyl)phenyl]benzamide;
2-[(4-Pyridyl)methyl]amino-N-[(3-methylsulphinylphenyl]benzamide;
2-[(4-Pyridyl)methyl]amino-N-[4-(1,1-dimethylethyl)phenyl]benzamide;
2-[(4-Pyridyl)methyl]amino-N-(3-chlorophenyl)benzamide;
2-[(4-Pyridyl)methyl]amino-N-(3-bromophenyl)benzamide;
2-[(4-Pyridyl)methyl]amino-N-(3-methylphenyl)benzamide;
2-[(4-Pyridyl)methyl]amino-N-(3-benzoylphenyl)benzamide;
2-[(4-Pyridyl)methyl]amino-N-[3-(aminocarbonyl)phenyl]benzamide;
2-[(3-Pyridyl)methyl]amino-N-[3-(trifluoromethyl)phenyl]benzamide;
2-[(4-Quinolinyl)methyl]amino-N-[3-(trifluoromethyl)phenyl]benzamide;
2-[(5-Quinolinyl)methyl]amino-N-[3-(trifluoromethyl)phenyl]benzamide;
2-[(4-(2-Methyl)pyridyl)methyl]amino-N-[3-(trifluoromethyl)phenyl]benzamide;
2-[(4-(1,2-Dihydro-2-oxo)pyridyl)methyl]amino-N-[3-(trifluoromethyl)phenyl]-benzamide;
2-[(4-Quinolinyl)methyl]amino-N-(4-chlorophenyl)benzamide;
2-[(2-imidazolyl)methyl]amino-N-(4-chlorophenyl)benzamide;
2-[2-(4-Pyridyl)ethyl]amino-N-[3-(trifluoromethyl)phenyl]benzamide;
2-[2-(3-Pyridyl)ethyl]amino-N-[3-(trifluoromethyl)phenyl]benzamide;
2-[1-Methyl-2-(3-pyridyl)ethyl]amino-N-[3-(trifluoromethyl)phenyl]benzamide;
2-[(1-Oxido-4-pyridyl)methyl]amino-N-[3-(trifluoromethyl)phenyl]benzamide; and
2-[(4-Pyridyl)methyl]methylamino-N-[3-(trifluoromethyl)phenyl]benzamide;
2-[(4-Pyridyl)methyl]amino-N-(4-chloronaphthyl)benzamide;
6-Methyl-2-[(4-pyridyl)methyl]amino-N-(4-chlorophenyl)benzamide;
6-Chloro-2-[(4-pyridyl)methyl]amino-N-(4-chlorophenyl)benzamide;
3,4-Methylendioxy-6-[(4-pyridyl)methyl]amino-N-(4-chlorophenyl)benzamide;
4,5-Dimethyl-2-[(4-pyridyl)methyl]amino-N-(4-chlorophenyl)benzamide;
5-Chloro-2-[(4-pyridyl)methyl]amino-N-(4-n-propylphenyl)benzamide;
2-[(4-Pyridyl)methyl]amino-N-(4-n-propylphenyl)benzamide;
2-[(4-Pyridyl)methyl]amino-N-(7-hydroxynaphthyl)benzamide;
2-[(4-Pyridyl)methyl]amino-N-(8-hydroxy-2-naphthyl)benzamide;
4-Chloro-2-[(4-pyridyl)methyl]amino-N-(4-chlorophenyl)benzamide;
5-Methyl-2-[(4-pyridyl)methyl]amino-N-(4-chlorophenyl)benzamide;
2-[(4-Pyridyl)methyl]amino-N-(5,6,7,8-tetrahydronaphthyl)benzamide;
2-[(4-Pyridyl)methyl]amino-N-(4-biphenyl)benzamide;
5-Chloro-2-[(4-pyridyl)methyl]amino-N-(4-chlorophenyl)benzamide;
2-[(4-Pyridyl)methyl]amino-N-(naphthyl)benzamide;
2-[(4-Pyridyl)methyl]amino-N-(2-napthyl)benzamide;
2-[(4-Pyridyl)methyl]amino-N-(4-methoxyphenyl)benzamide;
2-[(4-Pyridyl)methyl]amino-N-[3-(trifluoromethoxy)phenyl]benzamide;
2-[(4-Pyridyl)methyl]amino-N-(4-methoxy-2-naphthyl)benzamide;
2-[(4-Pyridyl)methyl]amino-N-(3-bromo-2-naphthyl)benzamide;
2-[(4-Pyridyl)methyl]amino-N-[4-(isopropoxycarbonyl)phenyl]benzamide;
2-[(4-Pyridyl)methyl]amino-N-[4-(trifluoromethoxy)phenyl]benzamide;
2-[(4-Pyridyl)methyl]amino-N-[4-(isopropylcarbamoyl)phenyl]benzamide;
2-[(4-Pyridyl)methyl]amino-N-(3-chloro-4-methylphenyl)benzamide;
2-[(4-Pyridyl)methyl]amino-N-(2-methylphenyl)benzamide;
2-[(4-Pyridyl)methyl]amino-N-[3-(methoxycarbonylmethyl)phenyl]benzamide;
2-[(4-Pyridyl)methyl]amino-N-(4-phenoxyphenyl)benzamide;
   or a pharmaceutically acceptable salt thereof.

The most preferred compound of formula (II) is N-(4-chloro-3-trifluoromethyl-phenyl)-2-[(1-oxy-pyridin-4-ylmethyl)-amino]-benzamide or a pharmaceutically acceptable salt thereof.

Compounds of formula (II), and their preparation, are disclosed in WO 00/27820 published May 18, 2000 and U.S. Pat. No. 6,448,277, and are herewith incorporated.

Further VEGF inhibitors for use in the combinations of the present invention include those of formula (III)

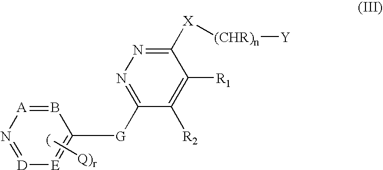

wherein
r is 0 to 2,
n is 0 to 2,
m is 0 to 4,
R₁ and R₂ (i) are lower alkyl or
(ii) together form a bridge in subformula (III*)

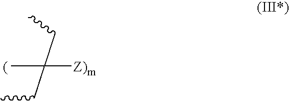

the binding being achieved via the two terminal carbon atoms, or
(iii) together form a bridge in subformula (III**)

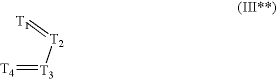

wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
   and wherein the bonds characterized, if present, by a wavy line are either single or double bonds;
   or an N-oxide of the defined compound, wherein 1 or more N atoms carry an oxygen atom,
   or a pharmaceutically acceptable salt of such compound having at least one salt-forming group.

The general terms used hereinbefore and hereinafter preferably have within the context of this disclosure for formula (III) the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms (for example in compounds of formula (III) [or an N-oxide thereof], wherein n=1 and R is lower alkyl) may be present in the (R)-, (S)- or (R,S)-configuration, preferably in the (R)- or (S)-configuration. Substituents at a double bond or a ring may be present in cis-(=Z-) or trans (=E-) form. The compounds may thus be present as mixtures of isomers or as pure isomers, preferably as enantiomer-pure diastereomers.

If R₁ and R₂ together form a bridge in subformula (III*), the pertinent compound of formula (III) has formula (IIIA) (compounds of this formula are hereinbefore and hereinafter especially preferred when compounds of formula (III) are mentioned),

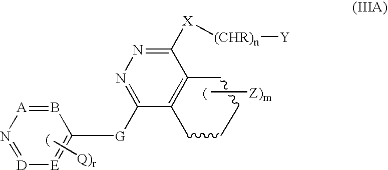

wherein the radicals are as defined above for compounds of formula (III).

If R₁ and R₂ together form a bridge in subformula (III**), the pertinent compound of formula (III) has formula (IIIB),

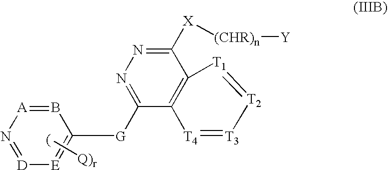

wherein the radicals are as defined above for compounds of formula (III).

Of the ring members T₁, T₂, T₃ and T₄, preferably only one is nitrogen, the remaining three being CH; preferably only T₃, especially T₄, is nitrogen, whereas the other ring members T₁, T₂, and T₄ or T₁, T₂, and T₃ are CH.

The index r is preferably 0 or 1.

The index n is preferably 0 or 1, especially 0.

The index m is preferably 0, 1, or 2, especially 0 or also 1.

Of ring members A, B, D, and E in formula (III), not more than 2 are N, and the remaining ones are CH. Preferably, each of the ring members A, B, D and E are CH.

If G is a bivalent group —CH₂O—, —CH₂—S—, or —CH₂—NH—, the methylene group in each case is bound to the ring with ring members A, B, D, and E, whereas the heteroatom (O, S, or NH) is bound to the phthalazine ring in formula (III).

Lower alkylene G may be branched or preferably linear and is especially branched or preferably linear C₁-C₄alkylene, especially methylene (—CH₂—), ethylene (—CH₂—CH₂—), trimethylene (—CH₂—CH₂—CH₂—) or tetramethylene (—CH₂—CH₂—CH₂—CH₂—). G is preferably methylene.

Acyl in lower alkylene substituted by acyloxy is preferably arylcarbonyloxy, wherein aryl is defined as below, especially benzoyloxy or lower alkanoyloxy, especially benzoyloxy; lower alkylene substituted by acyloxy is especially methylene substituted by benzoyloxy.

Lower alkylene substituted by hydroxy is preferably hydroxymethylene (—CH(OH)—).

G as lower alkylene substituted by acyloxy or hydroxy is preferred, or G as otherwise defined hereinbefore and hereinafter is in each case especially preferred.

Q is preferably bound to A or D (r=1) or to both (r=2), where in the event of binding of Q, A and/or D are/is C(-Q).

Lower alkyl is especially C₁-C₄-alkyl, e.g. n-butyl, sec-butyl, tert-butyl, n-propyl, isopropyl, or especially methyl or also ethyl.

In the preferred embodiment, aryl is an aromatic radical having 6 to 14 carbon atoms, especially phenyl, naphthyl, fluorenyl or phenanthrenyl, the radicals defined above being unsubstituted or substituted by one or more, preferably up to three, especially one or two substituents, especially selected from amino, mono- or disubstituted amino, halogen, Alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkylsulfonyl, and alkylphenylsulfonyl, or (as an alternative or in addition to the above group of substituents) selected from lower alkenyl, such as ethenyl, phenyl, lower alkylthio, such as methylthio, lower alkanoyl, such as acetyl, lower alkylmercapto, such as methylmercapto (—S—CH₃), halogen-lower alkylmercapto, such as trifluoromethylmercapto (—S—CF₃), lower alkylsulfonyl, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy; aryl is preferably phenyl which is either unsubstituted or independently substituted by one or two substituents selected from the group comprising amino; lower alkanoylamino, especially acetylamino; halogen, especially fluorine, chlorine, or bromine; lower alkyl, especially methyl or also ethyl or propyl; halogen-lower alkyl, especially trifluoromethyl; hydroxy; lower alkoxy, especially methoxy or also ethoxy; phenyl-lower alkoxy, especially benzyloxy; and cyano, or (as an alternative or in addition to the previous group of substituents) C₈-C₁₂alkoxy, especially n-decyloxy, carbamoyl, lower alkylcarbamoyl, such as n-methyl- or n-tert-butylcarbamoyl, lower alkanoyl, such as acetyl, phenyloxy, halogen-lower alkyloxy, such as trifluoromethoxy or 1,1,2,2-tetrafluoroethyloxy, lower alkoxycarbonyl, such as ethoxycarbonyl, lower alkylmercapto, such as methylmercapto, halogen-lower alkylmercapto, such as trifluoromethylmercapto, hydroxy-lower alkyl, such as hydroxymethyl or 1-hydroxymethyl, lower alkylsulfonyl, such as methane sulfonyl, halogen-lower alkylsulfonyl, such as trifluoromethane sulfonyl, phenylsulfonyl, dihydroxybora (—B(OH)₂), 2-methylpyrimidin-4-yl, oxazol-5-yl, 2-methyl-1,3-dioxolan-2-yl, 1H-pyrazol-3-yl, 1-methyl-pyrazol-3-yl and lower alkylene dioxy bound to two adjacent C-atoms, such as methylene dioxy.

Where mention is made hereinbefore and hereinafter to radicals or substituents as “an alternative or in addition to” the previous group of radicals or substituents, these radicals or substituents and those of the previous group are to be regarded together as one group of substituents from which the respective radicals may be selected, or especially as separate groups. The expression does not mean that one of the radicals following the expression may be added to a member of the previous group by binding. This applies, even if the expression “as an alternative or in addition to” is not mentioned again, for the radicals or substituents, as defined here, in the preferred compounds of formula (III) defined below.

Mono- or disubstituted amino is especially amino substituted by one or two radicals selected independently of one another from lower alkyl, such as methyl; hydroxy-lower alkyl, such as 2-hydroxyethyl; phenyl-lower alkyl; lower alkanoyl, such as acetyl; benzoyl; substituted benzoyl, wherein the phenyl radical is unsubstituted or especially substituted by one or more, preferably one or two, substituents selected from nitro or amino, or also from halogen, amino, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro or amino, or also from halogen, amino, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl or carbamoyl, or as an alternative or in addition to the previous group of radicals by aminocarbonylamino.

Halogen is especially fluorine, chlorine, bromine, or iodine, especially fluorine, chlorine, or bromine.

In the preferred embodiment, alkyl has up to a maximum of 12 carbon atoms and is especially lower alkyl, especially methyl, or also ethyl, n-propyl, isopropyl, or tert-butyl.

Substituted alkyl is alkyl as last defined, especially lower alkyl, preferably methyl; where one or more, especially up to three, substituents may be present, primarily from the group selected from halogen, especially fluorine, and also from amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Etherified hydroxy is especially C₈-C₂₀alkyloxy, such as n-decyloxy, lower alkoxy (preferred), such as methoxy, ethoxy, isopropyloxy, or n-pentyloxy, phenyl-lower alkoxy, such as benzyloxy, or also phenyloxy, or as an alternative or in addition to the previous group C₈-C₂₀alkyloxy, such as n-decyloxy, halogen-lower alkoxy, such as trifluoromethyloxy or 1,1,2,2-tetrafluoroethoxy.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxcarbonyloxy.

Esterified carboxy is especially lower alkoxycarbonyl, such as tert-butoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl.

N-mono- or N,N-disubstituted carbamoyl is especially substituted by one or two substituents, lower alkyl, phenyl-lower alkyl, or hydroxy-lower alkyl, at the terminal nitrogen atom.

Alkylphenylthio is especially lower alkylphenylthio.

Alkylphenylsulfinyl is especially lower alkylphenylsulfinyl.

Pyridyl Y is preferably 3- or 4-pyridyl.

Z is preferably amino, hydroxy-lower alkylamino, such as 2-hydroxyethylamino, lower alkanoylamino, such as acetylamino, nitrobenzoylamino, such as 3-nitrobenzoylamino, aminobenzoylamino, such as 4-aminobenzoylamino, phenyl-lower alkoxycarbonylamino, such as benzyloxycarbonylamino, or halogen, such as bromine; preferably only one substituent is present (m=1), especially one of the last mentioned, especially halogen. A compound of formula (III) (or an N-oxide thereof), wherein Z is absent (m=0), is quite especially preferred.

Unsubstituted or substituted cycloalkyl is preferably C₃-C₈cycloalkyl, which is unsubstituted or substituted in the same way as aryl, especially as defined for phenyl. Cyclohexyl or also cyclopentyl or cyclopropyl are preferred.

Heterocyclyl is especially a five or six-membered heterocyclic system with 1 or 2 heteroatoms selected from the group comprising nitrogen, oxygen, and sulfur, which may be unsaturated or wholly or partly saturated, and is unsubstituted or substituted especially by lower alkyl, such as methyl; a radical selected from 2-methylpyrimidin-4-yl, oxazol-5-yl, 2-methyl-1,3-dioxolan-2-yl, 1H-pyrazol-3-yl, and 1-methyl-pyrazol-3-yl is preferred.

Aryl in the form of phenyl which is substituted by lower alkylene dioxy bound to two adjacent C-atoms, such as methylenedioxy, is preferably 3,4-methylenedioxyphenyl.

The bonds in formula (III*) and (IIIA) characterized by wavy lines are present either as single or as double bonds. Preferably both are at the same time either single or double bonds.

An N-oxide of a compound of formula (III) is preferably an N-oxide in which a phthalazine-ring nitrogen or a nitrogen in the ring with ring members A, B, D, and E carries an oxygen atom, or several of the said nitrogen atoms carry an oxygen atom.

Salts are especially the pharmaceutically acceptable salts of compounds of formula (III) (or an N-oxide thereof.

VEGF inhibitors of formula (III) and their preparation are disclosed in WO 98/35958, published on Aug. 20, 1998, and are herewith incorporated.

Preference is given to a compound of formula (III) selected from the specific Examples disclosed in WO 98/35958.

The most preferred VEGF inhibitor for use according to the present invention is the compound of formula (III) with the chemical name 1-(4-chloroanilino)-4-(4-pyridylmethyl)phthalazine (other names: Vatalanib, PTK787 or ZK 222584) or pharmaceutically acceptable salts thereof, especially the succinate salt.

Other VEGF inhibitors suitable for use in the present invention include compounds, proteins or antibodies generically and specifically disclosed in WO 03/040101, WO 03/040102, WO 00/09495, WO 00/27820, WO 00/59509, WO 98/11223, WO 00/27819 and EP 0 769 947; those as described by Prewett et al., Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; SU11248; CEP-7055; CP-547,632; GW2286; PD 173074; or anti-VEGF antibodies or anti-VEGF receptor antibodies, e.g., bevacizumab (AVASTIN), HuMV833, IMC-1C11 and ranibizumab (RhuFab); VEGF aptamer, e.g., Macugon; and Angiozyme (RPI 4610).

### The Combinations

Thus, in a first aspect, the present invention relates to a method for the prevention or treatment of proliferative diseases or diseases that are triggered by persistent angiogenesis in a mammal, preferably a human patient, which comprises treating the patent concurrently or sequentially with pharmaceutically effective amounts of a combination of:
(a) a VEGF inhibitor compound, preferably of formula (I), (II) or (III); and
(b) one or more chemotherapeutic agents.

In another aspect, the present invention relates to a pharmaceutical composition comprising a combination of:
(a) a VEGF inhibitor compound, preferably of formula (I), (II) or (III); and
(b) one or more chemotherapeutic agents.

In a yet further aspect, the present invention provides a pharmaceutical composition comprising:
(a) a VEGF inhibitor compound, preferably of formula (I), (II) or (III); and
(b) one or more chemotherapeutic agents, together with a pharmaceutically acceptable carrier.

In a preferred embodiment, the present invention relates to a COMBINATION OF THE INVENTION comprising:
(a) a VEGF inhibitor compound, preferably of formula (I), (II) or (III); and
(b) one or more chemotherapeutic agents selected from the group consisting of HDAC inhibitors, microtube active agents, inhibitors or the EGF receptor tyrosine kinase family, mTOR inhibitors, COX-2 inhibitors, ionizing radiation, IGF-IR inhibitors, aromatase inhibitors, bisphosphonates, Bcr-Abl kinase inhibitors, FLT-3 kinase inhibitors, ALK inhibitors, c-Kit inhibitors, platelet-derived growth factor receptor inhibitors, Raf kinase inhibitors, HSP-90 inhibitors, antibodies against VEGF and VEGFR, MMP inhibitors, SRC inhibitors, farnesyl transferse inhibitors and EDG binders.

In another preferred embodiment, the present invention relates to a COMBINATION OF THE INVENTION comprising:
(a) a VEGF inhibitor compound, preferably of formula (I), (II) or (III); and
(b) one or more chemotherapeutic agents selected from the group consisting of N-hydroxy-3-[4-[[[2-(2-methyl-1H-indol-3-yl)-ethyl]-amino]methyl]phenyl]-2E-2-propenamide, or a pharmaceutically acceptable salt thereof, N-hydroxy-3-[4-[(2-hydroxyethyl){2-(1H-indol-3-yl)ethyl]-amino]methyl]phenyl}-2E-2-propenamide, or a pharmaceutically acceptable salt thereof, epothilones and derivatives thereof, taxanes, discodermolides, vinca alkaloids, colchicines, gefitinib, IGF-IR inhibitors, trastuzumab, RAD001, CCI-779, rapamycin, AP23573, lumiracoxib, celecoxib, valdecoxib, rofecoxib, 5FU, platin compounds, DNA alkylators, letrozole, anastrozole, exemestane, zoledronic acid, pamidronic acid, imatinib such as especially imatinib mesylate, PD173955, PKC412, MLN518, interferons, Ara-C, bisulfan, SU101, SU6668, GFB-111, BAY43-9006, PD184352,17-MG, geldanamycin-related compounds and radicicol.

Any of the combination of components (a) and (b), the method of treating a warm-blooded animal comprising administering these two components, a pharmaceutical composition comprising these two components for simultaneous, separate or sequential use, the use of the combination for the delay of progression or the treatment of a proliferative disease or for the manufacture of a pharmaceutical preparation/composition for these purposes or a commercial product comprising such a combination of components (a) and (b), all as mentioned or defined, is referred to herein also as COMBINATION OF THE INVENTION (so that this term refers to each of these embodiments which thus can replace this term where appropriate).

Simultaneous administration may, e.g., take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points, preferably meaning that the components (a) and (b) are administered such that no overlap of measurable blood levels of both compounds are present in an overlapping manner (at the same time).

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

“Jointly therapeutically active” or “joint therapeutic effect” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect). Whether this is the case, can inter alia be determined by following the blood levels, showing that both compounds are present in the blood of the human to be treated at least during certain time intervals.

“Pharmaceutically effective” preferably relates to an amount that is therapeutically or in a broader sense also prophylactically effective against the progression of a proliferative disease.

The term “a commercial package” or “a product”, as used herein defines especially a “kit of parts” in the sense that the components (a) and (b) as defined above can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal anti-tumor effect) or the like.

The COMBINATION OF THE INVENTION can also be applied in combination with other treatments, e.g., surgical intervention, hyperthermia and/or irradiation therapy.

The pharmaceutical compositions according to the present invention can be prepared by conventional means and are those suitable for enteral, such as oral or rectal, and parenteral administration to mammals including man, comprising a therapeutically effective amount of a VEGF inhibitor and at least one chemotherapeutic agent alone or in combination with one or more pharmaceutically acceptable carriers, especially those suitable for enteral or parenteral application.

The pharmaceutical compositions comprise from about 0.00002 to about 100%, especially, e.g., in the case of infusion dilutions that are ready for use, of 0.0001 to 0.02%, or, e.g., in case of injection or infusion concentrates or especially parenteral formulations, from about 0.1% to about 95%, preferably from about 1% to about 90%, more preferably from about 20% to about 60%, active ingredient (weight by weight, in each case). Pharmaceutical compositions according to the invention may be, e.g., in unit dose form, such as in the form of ampoules, vials, dragées, tablets, infusion bags or capsules.

The effective dosage of each of the combination partners employed in a formulation of the present invention may vary depending on the particular compound or pharmaceutical compositions employed, the mode of administration, the condition being treated and the severity of the condition being treated. A physician, clinician or veterinarian of ordinary skill can readily determine the effective amount of each of the active ingredients necessary to prevent, treat or inhibit the progress of the condition.

In the instance where the chemotherapeutic agent is selected from the group consisting of DNA topoisomerase I inhibitors; DNA topoisomerase II inhibitors; microtubule active agents; and anti-metabolites including agents which are inhibitors of thymidine production, inhibitors of vascular endothethial growth factor, DNA demethylating agents or protein-tyrosine kinase inhibitors, such as, e.g., ADRIAMYCIN, discodermolides and epothilones, 5FU, Camptothecin, Imatinib mesylate (GLEEVEC/GLIVEC), 1-[4-chloroanilinol-4-[pyridylmethyl]-phthalazine succinate, 5-Aza dC (DECITABINE) and 5-AZACYTIDINE; pharmaceutically acceptable salts or solvates thereof; and pharmaceutically acceptable prodrug esters thereof; and the patient to be treated is a human, an appropriate dose of, e.g., ADRIAMYCIN is in the range from 100-1500 mg daily, e.g., 200-1000 mg/day, such as 200, 400, 500, 600, 800, 900 or 1000 mg/day, administered in one or two doses daily. 5-FU is administered at a appropriate dose in the range from 100-1500 mg daily, e.g., 200-1000 mg/day, such as 200, 400, 500, 600, 800, 900 or 1000 mg/day, administered in one or two doses daily. CAMPTOTHECIN is administered at a appropriate dose in the range from 100-1500 mg daily, e.g., 200-1000 mg/day, such as 200, 400, 500, 600, 800, 900 or 1000 mg/day, administered in one or two doses daily. 5-AZACYTIDINE is administered at a appropriate dose in the range from 100-1500 mg daily, e.g., 200-1000 mg/day, such as 200, 400, 500, 600, 800, 900 or 1000 mg/day, administered in one or two doses daily. Among the topoisomerase II inhibitors, DOXORUBICIN may be administered to a human in a dosage range varying from about 10-100 mg/m²/day, e.g., 25 or 75 mg/m²/day, e.g., as single dose; Epirubicin may be administered to a human in a dosage range varying from about 10-200 mg/m²/day; IDARUBICIN may be administered to a human in a dosage range varying from about 0.5-50 mg/m²/day, e.g., 8 mg/m²/day during three days; and MITOXANTRONE may be administered to a human in a dosage range varying from about 2.5-25 mg/m²/day, e.g., 10-14 mg/m²/day during 5-8 days.

FADROZOLE may be administered orally to a human in a dosage range varying from about 0.5 mg/day to about 10 mg/day, preferably from about 1 mg/day to about 2.5 mg/day. EXEMESTANE may be administered orally to a human in a dosage range varying from about 5 mg/day to about 200 mg/day, preferably from about 10 mg/day to about 25 mg/day, or parenterally from about 50-500 mg/day, preferably from about 100 mg/day to about 250 mg/day. FORMESTANE may be administered parenterally to a human in a dosage range varying from about 100-500 mg/day, preferably from about 250 mg/day to about 300 mg/day. ANASTROZOLE may be administered orally to a human in a dosage range varying from about 0.25-20 mg/day, preferably from about 0.5 mg/day to about 2.5 mg/day. TAMOXIFEN citrate may be administered to a human in a dosage range varying from about 1040 mg/day. VINBLASTINE (not highly recommended as secondary malignancies may occur) may be administered to a human in a dosage range varying from about 1.5-10 mg/m²/day. Vincristine sulfate may be administered parenterally to a human in a dosage range varying from about 0.025-0.05 mg/kg body weight·week. VINORELBINE may be administered to a human in a dosage range varying from about 10-50 mg/m²/day. PACLITAXEL may be administered to a human in a dosage range varying from about 50-300 mg/m² day. DOCETAXEL may be administered to a human in a dosage range varying from about 25-100 mg/m²/day. 5-FU may be administered to a human in a dosage range varying from about 50-1000 mg/m²/day, e.g., 500 mg/m²/day. CAPECITABINE may be administered to a human in a dosage range varying from about 10-1000 mg/m²/day. GEMCITABINE hydrochloride (not highly recommended as secondary malignancies may occur) may be administered to a human in a dosage range varying from about 1000 mg/week. METHOTREXATE may be administered to a human in a dosage range varying from about 5-500 mg/m²/day. IRINOTECAN may be administered to a human in a dosage range varying from about 50-350 mg/m²/day. CARBOPLATIN may be administered to a human in a dosage range varying from about 200-400 mg/m² about every four weeks. CISPLATIN may be administered to a human in a dosage range varying from about 25-75 mg/m² about every three weeks. OXALIPLATIN may be administered to a human in a dosage range varying from about 50-85 mg/m² every two weeks. Alendronic acid may be administered to a human in a dosage range varying from about 5-10 mg/day. Clodronic acid may be administered to a human, e.g., in a dosage range varying from about 750-1500 mg/day. Etridonic acid may be administered to a human in a dosage range varying from about 200400 mg/day. Ibandronic acid may be administered to a human in a dosage range varying from about 14 mg every 3-4 weeks. Risedronic acid may be administered to a human in a dosage range varying from about 20-30 mg/day. Pamidronic acid may be administered to a human in a dosage range varying from about 15-90 mg every 3-4 weeks. Tiludronic acid may be administered to a human in a dosage range varying from about 200400 mg/day. Trastuzumab may be administered to a human in a dosage range varying from about 14 mg/m²/week. Bicalutamide may be administered to a human in a dosage range varying from about 25-50 mg/m² day.

Tyrphostins, especially Adaphostin, are preferably administered to a warm-blooded animal, especially a human in a dosage in the range of about 1-6000 mg/day, more preferably 25-5000 mg/day, most preferably 50-4000 mg/day. Unless stated otherwise herein, the compound is preferably administered from one to 5, especially from 14 times per day.

Pharmaceutical preparations for the combination therapy for enteral or parenteral administration are, e.g., those in unit dosage forms, such as sugar-coated tablets, capsules or suppositories, and furthermore ampoules. If not indicated otherwise, these formulations are prepared by conventional means, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units. One of skill in the art has the ability to determine appropriate pharmaceutically effective amounts of the combination components.

Preferably, the compounds or the pharmaceutically acceptable salts thereof, are administered as an oral pharmaceutical formulation in the form of a tablet, capsule or syrup; or as parenteral injections if appropriate.

In preparing compositions for oral administration, any pharmaceutically acceptable media may be employed such as water, glycols, oils, alcohols, flavoring agents, preservatives, coloring agents. Pharmaceutically acceptable carriers include starches, sugars, microcrystalline celluloses, diluents, granulating agents, lubricants, binders, disintegrating agents.

Solutions of the active ingredient, and also suspensions, and especially isotonic aqueous solutions or suspensions, are useful for parenteral administration of the active ingredient, it being possible, e.g., in the case of lyophilized compositions that comprise the active ingredient alone or together with a pharmaceutically acceptable carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers, and are prepared in a manner known per se, e.g., by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes.

The isotonic agent may be selected from any of those known in the art, e.g. mannitol, dextrose, glucose and sodium chloride. The infusion formulation may be diluted with the aqueous medium. The amount of aqueous medium employed as a diluent is chosen according to the desired concentration of active ingredient in the infusion solution. Infusion solutions may contain other excipients commonly employed in formulations to be administered intravenously such as antioxidants.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a patient sub-population to be treated or the needs of the single patient based on the severity of any side effects that the patient experiences.

The present invention especially relates to a combined preparation which comprises:
(a) one or more unit dosage forms of a VEGF inhibitor; and
(b) one or more unit dosage forms of a chemotherapeutic agent.

### The Diseases to be Treated

The combinations of the present invention are useful for treating proliferative diseases or diseases that are associated with or triggered by persistent angiogenesis.

A proliferative disease is mainly a tumor, especially a solid tumor, disease (or cancer) (and/or any metastases). The combinations of the present invention are particularly useful for treating a breast tumor, genitourinary tumor, lung tumor, gastrointestinal tumor, epidermoid tumor, melanoma, glioma such as especially glioblastoma and in particular glioblastoma multiforme, ovarian cancer, pancreas cancer, neuroblastoma, head and/or neck tumor, bladder cancer, or renal cancer such as especially renal cell carcinoma, in particular, (i) breast cancer; an epidermoid tumor, such as epidermoid head and/or neck cancer or a mouth tumor; lung cancer, e.g., small cell or especially non-small cell lung cancer; a gastrointestinal tumor, such as especially colorectal cancer; or a genitourinary tumor, such as especially prostate cancer (in particular a hormone-refractory prostate cancer); or (ii) a proliferative disease that is refractory to the treatment with other chemotherapeutics; or (iii) a tumor that is refractory to treatment with other chemotherapeutics due to multidrug resistance.

The combinations of the present invention are also useful in the treatment of other hyperproliferative conditions (hyperplasias), such as leukemias, especially acute myeloid leukemia (AML) and myeloma, in particular multiple myeloma; myelodysplastic syndrome; mesothelioma; adenocarcinomas, such as especially colorectal and pancreatic adenocarcinomas; liver cancer, such as especially hepatocellular carcinoma; fibrosis (especially pulmonary, but also other types of fibrosis, such as renal fibrosis); psoriasis; arteriosclerosis; and smooth muscle cell proliferation in the blood vessels due to e.g. stenosis or restenosis following angioplasty.

The combinations of the present invention can also be used to prevent or treat diseases that are triggered by persistent angiogenesis, such as psoriasis; Kaposi's sarcoma; restenosis, e.g., stent-induced restenosis; endometriosis; Crohn's disease; Hodgkin's disease; arthritis, such as rheumatoid arthritis; hemangioma; angiofibroma; ocular diseases, such as ocular neovascularization, diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; injuries of the nerve tissue; for inhibiting the re-occlusion of vessels after balloon catheter treatment; for use in vascular prosthetics or after inserting mechanical devices for holding vessels open, such as, e.g., stents; as immunosuppressants; as an aid in scar-free wound healing; and for treating age spots and contact dermatitis.

The combinations of the present invention are further also useful for the treatment, prevention or inhibition of diseases characterized by cell proliferation and infiltration of inflammatory cells such as inflammation, rheumatoid arthritis, asthma, chronic bronchitis, arteriosclerosis, and transplant rejection.

The combinations of the present invention are also useful in the treatment of diseases which involve VEGFR driven, especially VEGFR-3 driven lymphangiogenesis.

Other malignancies which may be treated according to this invention includes a malignancy such as lymphoma and cancer of the esophagus, uterus or cervix.

Where a tumor, a tumor disease, a carcinoma or a cancer are mentioned, also metastasis in the original organ or tissue and/or in any other location are implied alternatively or in addition, whatever the location of the tumor and/or metastasis.

The combinations of the present invention can also be used to treat, inhibit or prevent c-kit indications, such as gastrointestinal stromal tumors (GIST), small cell lung cancer, dog mastocytosis and feline sarcoma viruses.

What is claimed is:

1. A method for the prevention or treatment of proliferative diseases, which comprises administering pharmaceutically effective amounts of a combination of:
(a) a VEGF inhibitor compound; and
(b) one or more chemotherapeutic agents selected from the group consisting of:
i. an aromatase inhibitor;
ii. an anti-estrogen, an anti-androgen (especially in the case of prostate cancer) or a gonadorelin agonist;
iii. a topoisomerase I inhibitor or a topoisomerase II inhibitor;
iv. a microtubule active agent, an alkylating agent, an anti-neoplastic anti-metabolite or a platin compound;
v. a compound targeting/decreasing a protein or lipid kinase activity or a protein or lipid phosphatase activity, a further anti-angiogenic compound or a compound which induces cell differentiation processes;
vi. a bradykinin 1 receptor or an angiotensin II antagonist;
vii. a cyclooxygenase inhibitor, a bisphosphonate, a heparanase inhibitor (prevents heparan sulphate degradation), e.g., PI-88, a biological response modifier, preferably a lymphokine or interferons, e.g., interferon γ, an ubiquitination inhibitor, or an inhibitor which blocks anti-apoptotic pathways;
viii. an inhibitor of Ras oncogenic isoforms or a farnesyl transferase inhibitor;
ix. a telomerase inhibitor, e.g., telomestatin;
x. a protease inhibitor, a matrix metalloproteinase inhibitor, a methionine aminopeptidase inhibitor, e.g., bengamide or a derivative thereof, or a proteasome inhibitor, e.g., PS-341;
xi. agents used in the treatment of hematologic malignancies or FMS-like tyrosine kinase inhibitors;
xii. an HSP90 inhibitors;
xiii. HDAC inhibitors;
xiv. mTOR inhibitors;
xv. Somatostatin receptor antagonists;
xvi. integrin antagonists;
xvii. antileukemic compounds;
xviii. tumor cell damaging approaches such as ionizing radiation;
xix. EDG binders;
xx. anthranilic acid amide class of kinase inhibitors;
xxi. ribonucleotide reductase inhibitors;
xxii. S-adenosylmethionine decarboxylase inhibitors;
xxiii. antibodies against VEGF or VEGFR;
xxiv. photodynamic therapy;
xxv. angiostatic steroids;
xxvi. implants containing corticosteroids;
xxvii. AT1 receptor antagonists; and
xxviii. ACE inhibitors.

2. The method according to claim 1, wherein the VEGF inhibitor compound is
(i) of the formula (I) 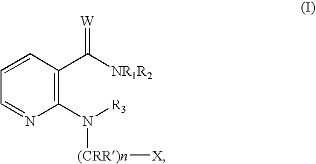
wherein
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
or of an N-oxide or a possible tautomer thereof;
or of a pharmaceutically acceptable salt;
(ii) of the formula (II) 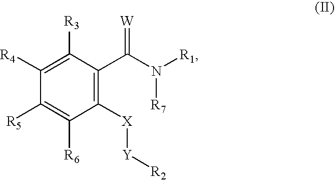
wherein
W is O or S;
X is NR₈;
Y is CR₉R₁₀—(CH₂)ₙ,
wherein
R₉ and R₁₀ are, independently, of each other hydrogen or lower alkyl; and
n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y=SO₂ cannot represent 2,1,3-benzothiadiazol-4-yl;
any of R₃, R₄, R₅ and R₈, independently of the other, is H or a substituent other than hydrogen; and
R₇ and R₈, independently of each other, are H or lower alkyl;
or of an N-oxide; or a pharmaceutically acceptable salt thereof; or
(iii) of the formula (III) 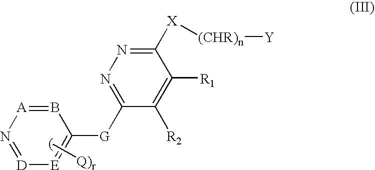
wherein
r is 0 to 2,
n is 0 to 2,
m is 0 to 4,
R₁ and R₂ (i) are lower alkyl or
(ii) together form a bridge in subformula (III*) 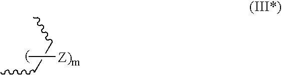
the binding being achieved via the two terminal carbon atoms, or
(iii) together form a bridge in subformula (III**) 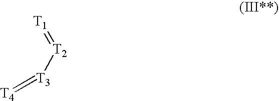
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or a pharmaceutically acceptable salt of such compound having at least one salt-forming group.

3. The method according to claim 1, wherein the VEGF inhibitor compound is 1-(4-chloroanilino)-4-(4-pyridylmethyl)phthalazine or a pharmaceutically acceptable salt thereof.

4. The method according to claim 1, which comprises administering pharmaceutically effective amounts of a combination of:
(a) a VEGF inhibitor compound; and
(b) one or more chemotherapeutic agents selected from the group consisting of HDAC inhibitors, microtube active agents, inhibitors or the EGF receptor tyrosine kinase family, mTOR inhibitors, COX-2 inhibitors, ionizing radiation, IGF-IR inhibitors, aromatase inhibitors, bisphosphonates, Bcr-Abl kinase inhibitors, FLT-3 kinase inhibitors, ALK inhibitors, c-Kit inhibitors, platelet-derived growth factor receptor inhibitors, Raf kinase inhibitors, HSP-90 inhibitors, antibodies against VEGF and VEGFR, MMP inhibitors, SRC inhibitors, farnesyl transferse inhibitors and EDG binders.

5. The method according to claim 4, wherein the VEGF inhibitor compound is
(i) of the formula (I) 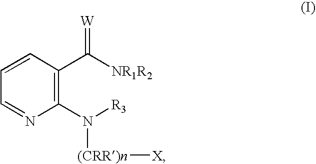
wherein
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
or of an N-oxide or a possible tautomer thereof;
or of a pharmaceutically acceptable salt;
(ii) of the formula (II) 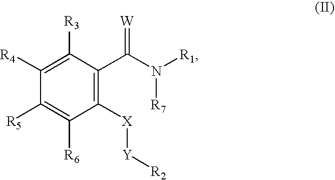
wherein
W is O or S;
X is NR₈;
Y is CR₉R₁₀—(CH₂)ₙ,
wherein
R₉ and R₁₀ are, independently, of each other hydrogen or lower alkyl; and
n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y=SO₂ cannot represent 2,1,3-benzothiadiazol-4-yl;
any of R₃, R₄, R₅ and R₆, independently of the other, is H or a substituent other than hydrogen; and
R₇ and R₈, independently of each other, are H or lower alkyl;
or of an N-oxide; or a pharmaceutically acceptable salt thereof; or
(iii) of the formula (III) 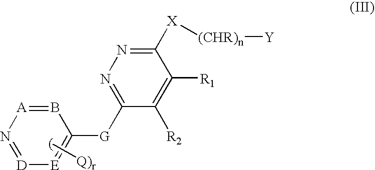
wherein
r is 0 to 2,
n is 0 to 2,
m is 0 to 4,
R₁ and R₂ (i) are lower alkyl or
(ii) together form a bridge in subformula (III*) 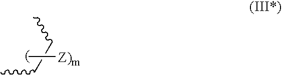
the binding being achieved via the two terminal carbon atoms, or
(iii) together form a bridge in subformula (III**) 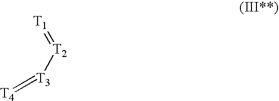
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or a pharmaceutically acceptable salt of such compound having at least one salt-forming group.

6. The method according to claim 4, wherein the VEGF inhibitor compound is 1-(4-chloroanilino)-4-(4-pyridylmethyl)phthalazine or a pharmaceutically acceptable salt thereof.

7. The method according to claim 1, which comprises administering pharmaceutically effective amounts of a combination of:
(a) a VEGF inhibitor compound, and
(b) one or more chemotherapeutic agents selected from the group consisting of N-hydroxy-3-[4-[[[2-(2-methyl-1H-indol-3-yl)-ethyl]-amino]methyl]phenyl]-2E-2-propenamide, or a pharmaceutically acceptable salt thereof, N-hydroxy-3-[4-[(2-hydroxyethyl){2-(1H-indol-3-yl)ethyl]-amino]methyl]phenyl]-2E-2-propenamide, or a pharmaceutically acceptable salt thereof, epothilones and derivatives thereof, taxanes, discodermolides, vinca alkaloids, colchicines, gefitinib, IGF-IR inhibitors, trastuzumab, RAD001, CCI-779, rapamycin, AP23573, lumiracoxib, celecoxib, valdecoxib, rofecoxib, 5-FU, platin compounds, DNA alkylators, letrozole, anastrozole, exemestane, zoledronic acid, pamidronic acid, imatinib such as especially imatinib mesylate, PD173955, PKC412, MLN518, interferons, Ara-C, bisulfan, SU101, SU6668, GFB-111, BAY43-9006, PD184352, 17-MG, geldanamycin-related compounds and radicicol.

8. The method according to claim 7, wherein the VEGF inhibitor compound is
(i) of the formula (I) 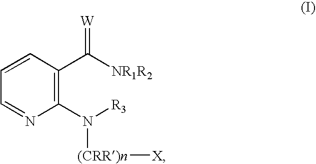
wherein
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
or of an N-oxide or a possible tautomer thereof;
or of a pharmaceutically acceptable salt;
(ii) of the formula (II) 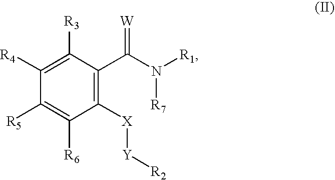
wherein
W is O or S;
X is NR₈;
Y is CR₉R₁₀—(CH₂)ₙ,
wherein
R₉ and R₁₀ are, independently, of each other hydrogen or lower alkyl; and
n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y═SO₂ cannot represent 2,1,3-benzothiadiazol-4-yl;
any of R₃, R₄, R₅ and R₆, independently of the other, is H or a substituent other than hydrogen; and
R₇ and R₈, independently of each other, are H or lower alkyl;
or of an N-oxide; or a pharmaceutically acceptable salt thereof; or
(iii) of the formula (III) 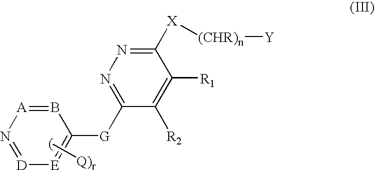
wherein
r is 0 to 2,
n is 0 to 2,
m is 0 to 4,
R₁ and R₂ (i) are lower alkyl or
(ii) together form a bridge in subformula (III*) 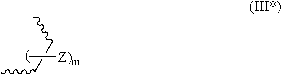
the binding being achieved via the two terminal carbon atoms, or
(iii) together form a bridge in subformula (III**) 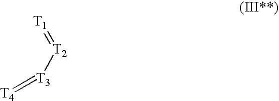
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or a pharmaceutically acceptable salt of such compound having at least one salt-forming group.

9. The method according to claim 7, wherein the VEGF inhibitor compound is 1-(4-chloroanilino)-4-(4-pyridylmethyl)phthalazine or a pharmaceutically acceptable salt thereof.

10. A pharmaceutical composition comprising:
(a) a VEGF inhibitor compound; and
(b) one or more chemotherapeutic agents selected from the group consisting of:
i. an aromatase inhibitor;
ii. an anti-estrogen, an anti-androgen (especially in the case of prostate cancer) or a gonadorelin agonist;
iii. a topoisomerase I inhibitor or a topoisomerase II inhibitor;
iv. a microtubule active agent, an alkylating agent, an anti-neoplastic anti-metabolite or a platin compound;
v. a compound targeting/decreasing a protein or lipid kinase activity or a protein or lipid phosphatase activity, a further anti-angiogenic compound or a compound which induces cell differentiation processes;
vi. a bradykinin 1 receptor or an angiotensin II antagonist;
vii. a cyclooxygenase inhibitor, a bisphosphonate, a heparanase inhibitor (prevents heparan sulphate degradation), e.g., PI-88, a biological response modifier, preferably a lymphokine or interferons, e.g., interferon γ, an ubiquitination inhibitor, or an inhibitor which blocks anti-apoptotic pathways;
viii. an inhibitor of Ras oncogenic isoforms or a farnesyl transferase inhibitor;
ix. a telomerase inhibitor, e.g., telomestatin;
x. a protease inhibitor, a matrix metalloproteinase inhibitor, a methionine aminopeptidase inhibitor, e.g., bengamide or a derivative thereof, or a proteasome inhibitor, e.g., PS-341;
xi. agents used in the treatment of hematologic malignancies or FMS-like tyrosine kinase inhibitors;
xii. an HSP90 inhibitors;
xiii. HDAC inhibitors;
xiv. mTOR inhibitors;
xv. somatostatin receptor antagonists;
xvi. integrin antagonists;
xvii. anti-leukemic compounds;
xviii. tumor cell damaging approaches, such as ionizing radiation;
xix. EDG binders;
xx. anthranilic acid amide class of kinase inhibitors;
xxi. ribonucleotide reductase inhibitors;
xxii. S-adenosylmethionine decarboxylase inhibitors;
xxiii. antibodies against VEGF or VEGFR;
xxiv. photodynamic therapy;
xxv. angiostatic steroids;
xxvi. implants containing corticosteroids;
xxvii. AT1 receptor antagonists; and
xxviii. ACE inhibitors.

11. The pharmaceutical composition according to claim 10, wherein the VEGF inhibitor compound is
(i) of the formula (I) 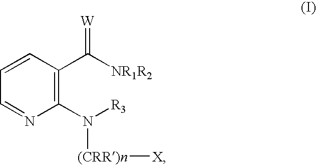
wherein
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
or of an N-oxide or a possible tautomer thereof;
or of a pharmaceutically acceptable salt;
(ii) of the formula (II) 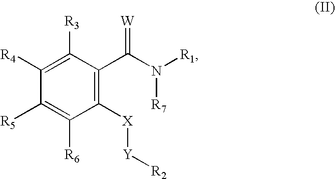
wherein
W is O or S;
X is NR₈;
Y is CR₉R₁₀—(CH₂)ₙ,
wherein
R₉ and R₁₀ are, independently, of each other hydrogen or lower alkyl; and
n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y=SO₂ cannot represent 2,1,3-benzothiadiazol-4-yl;
any of R₃, R₄, R₅ and R₆, independently of the other, is H or a substituent other than hydrogen; and
R₇ and R₈, independently of each other, are H or lower alkyl;
or of an N-oxide; or a pharmaceutically acceptable salt thereof; or
(iii) of the formula (III) 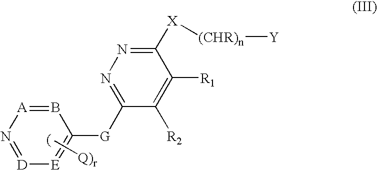
wherein
r is 0 to 2,
n is 0 to 2,
m is 0 to 4,
R₁ and R₂ (i) are lower alkyl or
(ii) together form a bridge in subformula (III*) 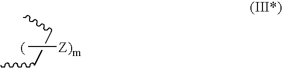
the binding being achieved via the two terminal carbon atoms, or
(iii) together form a bridge in subformula (III**) 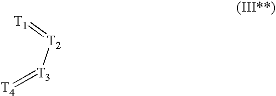
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or a pharmaceutically acceptable salt of such compound having at least one salt-forming group.

12. The pharmaceutical composition according to claim 10, wherein the VEGF inhibitor compound is 1-(4-chloroanilino)-4-(4-pyridylmethyl)phthalazine or a pharmaceutically acceptable salt thereof.

13. The pharmaceutical composition according to claim 10 comprising:
(a) a VEGF inhibitor compound; and
(b) one or more chemotherapeutic agents selected from the group consisting of HDAC inhibitors, microtube active agents, inhibitors or the EGF receptor tyrosine kinase family, mTOR inhibitors, COX-2 inhibitors, ionizing radiation, IGF-IR inhibitors, aromatase inhibitors, bisphosphonates, Bcr-Abl kinase inhibitors, FLT-3 kinase inhibitors, ALK inhibitors, c-Kit inhibitors, platelet-derived growth factor receptor inhibitors, Raf kinase inhibitors, HSP-90 inhibitors, antibodies against VEGF and VEGFR, MMP inhibitors, SRC inhibitors, farnesyl transferse inhibitors and EDG binders.

14. The pharmaceutical composition according to claim 13, wherein the VEGF inhibitor compound is
(i) of the formula (I) 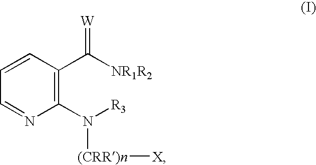
wherein
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
or of an N-oxide or a possible tautomer thereof;
or of a pharmaceutically acceptable salt;
(ii) of the formula (II) 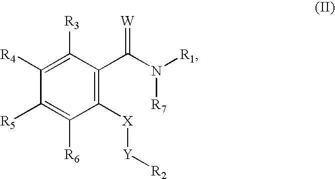
wherein
W is O or S;
X is NR₈;
Y is CR₉R₁₀—(CH₂)ₙ,
wherein
R₉ and R₁₀ are, independently, of each other hydrogen or lower alkyl; and
n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y=SO₂ cannot represent 2,1,3-benzothiadiazol-4-yl;
any of R₃, R₄, R₅ and R₆, independently of the other, is H or a substituent other than hydrogen; and
R₇ and R₈, independently of each other, are H or lower alkyl;
or of an N-oxide; or a pharmaceutically acceptable salt thereof; or
(iii) of the formula (III) 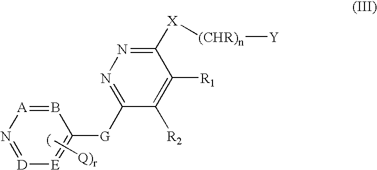
wherein
r is 0 to 2,
n is 0 to 2,
m is 0 to 4,
R₁ and R₂ (i) are lower alkyl or
(ii) together form a bridge in subformula (III*) 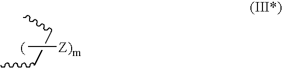
the binding being achieved via the two terminal carbon atoms, or
(iii) together form a bridge in subformula (III**) 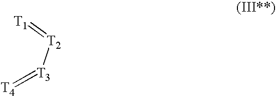
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or a pharmaceutically acceptable salt of such compound having at least one salt-forming group.

15. The pharmaceutical composition according to claim 13, wherein the VEGF inhibitor compound is 1-(4-chloroanilino)-4-(4-pyridylmethyl)phthalazine or a pharmaceutically acceptable salt thereof.

16. The pharmaceutical composition according to claim 10 comprising:
(a) a VEGF inhibitor compound; and
(b) one or more chemotherapeutic agents selected from the group consisting of N-hydroxy-3-[(4-[[[2-(2-methyl-1H-indol-3-yl)-ethyl]-amino]methyl]phenyl]-2E-2-propenamide, or a pharmaceutically acceptable salt thereof, N-hydroxy-3-[4-[(2-hydroxyethyl){2-(1H-indol-3-yl)ethyl]-amino]methyl]phenyl]-2E-2-propenamide, or a pharmaceutically acceptable salt thereof, epothilones and derivatives thereof, taxanes, discodermolides, vinca alkaloids, colchicines, gefitinib, IGF-IR inhibitors, trastuzumab, RAD001, CCI-779, rapamycin, AP23573, lumiracoxib, celecoxib, valdecoxib, rofecoxib, 5-FU, platin compounds, DNA alkylators, letrozole, anastrozole, exemestane, zoledronic acid, pamidronic acid, imatinib such as especially imatinib mesylate, PD173955, PKC412, MLN518, interferons, Ara-C, bisulfan, SU101, SU6668, GFB-111, BAY43-9006, PD184352,17-AAG, geldanamycin-related compounds and radicicol.

17. The pharmaceutical composition according to claim 16, wherein the VEGF inhibitor compound is
(i) of the formula (I) 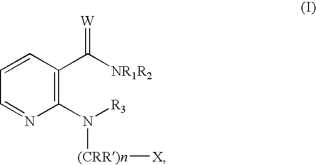
wherein
n is from 1 up to and including 6;
W is O or S;
R₁ and R₃ represent independently of each other hydrogen, lower alkyl or lower acyl;
R₂ represents an cycloalkyl group, an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or polysubstituted;
R and R′ are independently of each other hydrogen or lower alkyl; and
X represents an aryl group, or a mono- or bicyclic heteroaryl group comprising one or more ring nitrogen atoms and 0, 1 or 2 heteroatoms independently from each other selected from the group consisting of oxygen and sulfur, which groups in each case are unsubstituted or mono- or poly-substituted;
or of an N-oxide or a possible tautomer thereof;
or of a pharmaceutically acceptable salt;
(ii) of the formula (II) 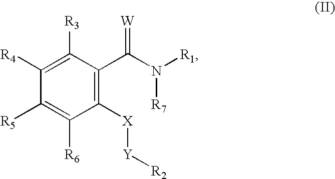
wherein
W is O or S;
X is NR₈;
Y is CR₉R₁₀—(CH₂)ₙ,
wherein
R₉ and R₁₀ are, independently, of each other hydrogen or lower alkyl; and
n is an integer of from and including 0 to and including 3; or
Y is SO₂;
R₁ is aryl;
R₂ is a mono- or bicyclic heteroaryl group comprising one or more ring nitrogen atoms with the exception that R₂ cannot represent 2-phthalimidyl, and in case of Y═SO₂ cannot represent 2,1,3-benzothiadiazol-4-yl;
any of R₃, R₄, R₅ and R₆, independently of the other, is H or a substituent other than hydrogen; and
R₇ and R₈, independently of each other, are H or lower alkyl;
or of an N-oxide; or a pharmaceutically acceptable salt thereof; or
(iii) of the formula (III) 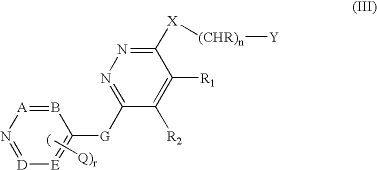
wherein
r is 0 to 2,
n is 0 to 2,
m is 0 to 4,
R₁ and R₂ (i) are lower alkyl or
(ii) together form a bridge in subformula (III*) 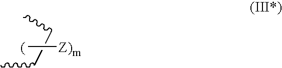
the binding being achieved via the two terminal carbon atoms, or
(iii) together form a bridge in subformula (III**) 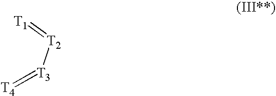
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or a pharmaceutically acceptable salt of such compound having at least one salt-forming group.

18. The pharmaceutical composition according to claim 16, wherein the VEGF inhibitor compound is 1-(4-chloroanilino)-4-(4-pyridylmethyl)phthalazine or a pharmaceutically acceptable salt thereof.

19. The method of claim 1, wherein the proliferative disease is selected from the group consisting of breast cancer, lung cancer, ovarian cancer, lymphoma, head and/or neck cancer and cancer of the esophagus, stomach, bladder, prostrate, uterus and cervix.
